(19) United States
(12) Patent Application Publication
(10) Pub. No.: US 2026/0226060 A1
(43) Pub. Date: Aug. 6, 2026

(54) SALTS AND CRYSTALLINE FORMS OF COMPOUND A, METHODS OF PREPARATION, AND USES THEREOF

(71) Applicant: SHENZHEN NEWDEL BIOTECH CO., LTD., Shenzhen, Guangdong (CN)

(72) Inventors: Xun HE, Shenzhen, Guangdong (CN); Feng JIN, Shenzhen, Guangdong (CN); Liye HUANG, Shenzhen, Guangdong (CN); Yuhan GUO, Shenzhen, Guangdong (CN); Tianyuan ZHANG, Shenzhen, Guangdong (CN); Zhenwei LI, Shenzhen, Guangdong (CN)

(73) Assignee: SHENZHEN NEWDEL BIOTECH CO., LTD., Shenzhen, Guangdong (CN)

(21) Appl. No.: 19/147,319

(22) PCT Filed: Jan. 12, 2024
(86) PCT No.: PCT/CN2024/071957

§ 371 Date: Jul. 10, 2025

(30) Foreign Application Priority Data
Jan. 13, 2023 (CN) 202310084790.5

(51) Int. Cl. C07D 487/04

(52) U.S. Cl. CPC C07D 487/04

(57) ABSTRACT

The present invention relates to pharmaceutically acceptable salts of N-(3-chloro-5-(trifluoromethyl)phenyl)-3-((6-(4-hydroxypiperidin-1-yl)imidazo[1,2-b]pyridazin-3-yl)ethynyl)-2-methylbenzamide, and crystalline forms and preparation methods thereof.

### TECHNICAL FIELD

The present invention relates to pharmaceutically acceptable salts of N-(3-chloro-5-(trifluoromethyl)phenyl)-3-((6-(4-hydroxypiperidin-1-yl)imidazo[1,2-b]pyridazin-3-yl)ethynyl)-2-methylbenzamide, and crystalline forms and preparation methods thereof.

### BACKGROUND

Tropomyosin receptor kinase (TRK) is a receptor tyrosine kinase (RTK) family, the members are TRKA, TRKB, and TRKC, encoded by the NTRKI, NTRK2, and NTRK3 genes, respectively. TRK is a class of transmembrane proteins consisting of an extracellular ligand-binding region, a transmembrane domain (TM) and an intracellular region, and activates mainly by binding neurotrophic factors (NTs). Neurotrophic factor is a class of protein molecules produced by nerve-innervated tissues (e.g., muscles) and astrocytes that are essential for neuronal growth and survival. At present, four major neurotrophic factors have been discovered, namely NGF (nerve growth factors), BDNF (brain-derived neurotrophic factor), NT-3 (neurotrophic factor 3) and NT-4 (neurotrophic factor 4). Wherein NGF binds to TRKA, BDNF and NT-4 bind to TRKB, and NT-3 can bind to all three TRK proteins, but it binds more strongly to TRKC. When activated by signal induction, TRK activates downstream signaling pathways sequentially through self-dimerization and phosphorylation to achieve various cellular physiological functions. The downstream signaling pathways of TRK include MAPK, PI3K/AKT, and PLCγ/PKC pathways, these signaling pathways regulate cell proliferation, differentiation, migration, apoptosis and other physiological processes, as well as a variety of neuron-related physiological activities, such as neurosynaptic flexibility, neural dendrite growth and repair, prevention and repair of neuronal degradation and maintenance of sensory neurons.

Numerous studies have shown that TRK overexpression, gene fusions, and mononucleotide alterations are closely related to the occurrence and development of various types of tumors, such as non-small cell lung cancer, breast cancer, colon cancer, prostate cancer, thyroid cancer, malignant melanoma, neuroblastoma, and mammary analog secretory carcinoma. Among mechanisms of abnormal TRK activation, the most prevalent mechanism is the gene fusion of TRK. The earliest NTRK fusion gene discovered in medical research was the TPM3-NTRK1 fusion gene found in colon cancer samples. With the deepening of research, researchers successively discovered multiple types of fusion genes, such as CD74-NTRK1, ETV6-NTRK2, QKI-NTRK2, and ETV6-NTRK3. The TRK fusion proteins expressed by NTRK fusion genes can continuously activate downstream signaling pathways independent of binding to ligands, thereby inducing abnormal cell proliferation and promoting the occurrence and development of tumors. Therefore, TRK is considered to be an effective target for anticancer therapy.

At present, a selective TRK inhibitor, Larotrectinib, developed by LOXO Inc. in USA was approved by the FDA in 2018; and a TRK inhibitor, Entrectinib, developed by Roche Pharmaceuticals Inc. was launched in Japan in June 2019. Belizatinib developed by TESARO Inc. is undergoing clinical study. In addition, multi-target inhibitors such as Cabozanitinib, Sitravatinib and Altiratinib also have good TRK inhibitory activity.

The NTRK gene point mutation caused by continuous use of TRK inhibitors is the key factor of drug resistance in tumors. Clinical studies have successively discovered mutations on G595R, G667C, F589L, and G667S in NTRK1, and on G623R and G696A in NTRK3. There is currently no inhibitor targeting these mutations on the market, and the second-generation TRK inhibitors, LOXO-195, TPX-0005, and ONO-5390556 are under clinical investigation.

N-(3-chloro-5-(trifluoromethyl)phenyl)-3-((6-(4-hydroxypiperidin-1-yl)imidazo [1,2-b]pyridazin-3-yl)ethynyl)-2-methylbenzamide (named as Compound A) is a TRK protein kinase inhibitor, it can effectively inhibit the activity of TRK protein kinase and inhibit the proliferation, migration and invasion of a variety of tumor cells, especially, it also has good pharmacokinetic properties and anti-drug resistance, and its structure is shown in the following Formula 1.

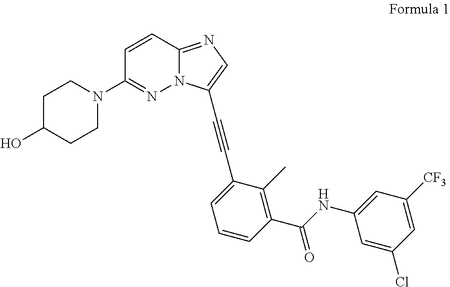 [SMILES: Cc1c(C#Cc2cnc3ccc(N4CCC(O)CC4)nn23)cccc1C(=O)Nc1cc(Cl)cc(C(F)(F)F)c1]

### SUMMARY OF THE INVENTION

A first aspect of the present invention relates to pharmaceutically acceptable salts of N-(3-chloro-5-(trifluoromethyl)phenyl)-3-((6-(4-hydroxypiperidin-1-yl) imidazo[1,2-b]pyridazin-3-yl)ethynyl)-2-methylbenzamide, wherein the pharmaceutically acceptable salt is an inorganic acid addition salt or an organic acid addition salt that is conventional in the art, further, the inorganic acid addition salt is preferably hydrochloride, sulfate or phosphate; the organic acid addition salt is preferably p-toluenesulfonate, mesylate, besylate, oxalate, maleate, L-camphor sulfonate, gentianate, tartrate or fumarate. The pharmaceutically acceptable salt of compound A is preferably phosphate or fumarate, and more preferably fumarate. In compound A phosphate, the ratio of compound A to phosphoric acid is preferably 1:1. In compound A fumarate, the ratio of compound A to fumaric acid is preferably 1:0.5.

A second aspect of the present invention relates to preparation of pharmaceutically acceptable salts of N-(3-chloro-5-(trifluoromethyl)phenyl)-3-((6-(4-hydroxypiperidin-1-yl)imidazo[1,2-b]pyridazin-3-yl)ethynyl)-2-methylbenzamide, the compound can be prepared according to the conventional salification methods in the art, comprising contacting compound A with the corresponding acid under conditions suitable for the formation of the corresponding acid addition salt.

A third aspect of the present invention relates to various crystalline forms of pharmaceutically acceptable salts of N-(3-chloro-5-(trifluoromethyl)phenyl)-3-((6-(4-hydroxypiperidin-1-yl)imidazo[1,2-b]pyridazin-3-yl)ethynyl)-2-methylbenzamide, including the following hydrochloride crystalline forms I and II, sulfate crystalline forms I, II, III and IV, p-toluenesulfonate crystalline forms I, II, III and IV, mesylate crystalline form I, besylate crystalline forms I and II, oxalate crystalline form I, maleate crystalline form I, phosphate crystalline forms I, II and III, L-camphorsulfonate crystalline forms I and II, tartrate crystalline form I and fumarate crystalline form I, preferably phosphate crystalline form I and fumarate crystalline form I, more preferably fumarate crystalline form I.

In compound A phosphate crystalline form I, the ratio of compound A to phosphoric acid is preferably 1:1. Compound A phosphate crystalline form I has one, more or all of the following characteristic XRPD peaks in terms of 2θ±0.2°: 4.339°, 6.033°, 6.651°, 7.308°, 8.631°, 10.392°, 10.640°, 12.690°, 12.914°, 14.147°, 14.437°, 15.026°, 17.457°, 17.890°, 18.468°, 19.006°, 19.487°, 19.938°, 20.266°, 20.846°, 21.619°, 21.987°, 22.604°, 23.640°, 23.875, 24.484°, 25.113°, 25.388°, 25.979°, 26.438°, 27.252°, 27.580°, 27.976°, 28.434°, 29.326°, 30.441°, 30.914°, 31.363°, 32.269°, 32.556°, 34.144°, 34.722°, 35.890°, 36.732°, 37.404°, 38.730°, 39.149°.

One aspect, the phosphate crystalline form I has at least one characteristic XRPD peak in terms of 2θ selected from 4.3°±0.2°, 17.4°±0.2°, 21.6°±0.2° and 21.9°±0.2°; preferably, the phosphate crystalline form I has characteristic XRPD peaks in terms of 2θ: 4.3°±0.2°, 17.4°±0.2°, 21.6°±0.2° and 21.9°±0.2°.

Another aspect, the phosphate crystalline form I has at least one characteristic XRPD peak in terms of 2θ selected from 12.9°±0.2°, 14.4°±0.2°, 20.3°±0.2° and 22.6°±0.2°; preferably, the phosphate crystalline form I has characteristic XRPD peaks in terms of 2θ: 12.9°±0.2°, 14.4°±0.2°, 20.3°±0.2° and 22.6°±0.2°.

Another aspect, the phosphate crystalline form I has at least one characteristic XRPD peak in terms of 2θ selected from 12.7°±0.2°, 14.1°±0.2°, 19.9°±0.2° and 26.4°±0.2°; preferably, the phosphate crystalline form I has characteristic XRPD peaks in terms of 2θ: 12.7°±0.2°, 14.1°±0.2°, 19.9°±0.2° and 26.4°±0.2°.

Another aspect, the phosphate crystalline form I has at least one characteristic XRPD peak in terms of 2θ selected from 4.3°±0.2°, 17.4°±0.2°, 21.6°±0.2°, 21.9°±0.2°, 12.9°±0.2°, 14.4°±0.2°, 20.3°±0.2°, 22.6°±0.2°, 12.7°±0.2°, 14.1°±0.2°, 19.9°±0.2° and 26.4°±0.2°; preferably, the phosphate crystalline form I has characteristic XRPD peaks in terms of 2θ: 4.3°±0.2°, 17.4°±0.2°, 21.6°±0.2°, 21.9°±0.2°, 12.9°±0.2°, 14.4°±0.2°, 20.3°±0.2°, 22.6θ±0.2°, 12.7°±0.2°, 14.1°±0.20θ, 19.9°±0.2° and 26.4°±0.2°.

Another aspect, the phosphate crystalline form I has an XRPD pattern substantially as depicted in FIG. 19.

The present invention also provides a preparation method of the above compound A phosphate crystalline form I, comprising the following steps:
contacting compound A with phosphoric acid in a solvent to form compound A phosphate;
precipitating compound A phosphate as compound A phosphate form I.

In the above method, the solvent is isopropanol.

In the above method, the solvent is acetonitrile/water, and the ratio of acetonitrile to water is preferably ranging from 15:1 to 25:1, such as 16:1, 17:1, 18:1, 19:1, 20:1, 21:1, 22:1, 23:1, 24:1.

In the above method, the precipitation is carried out at room temperature.

In compound A fumarate crystalline form I, the ratio of compound A to fumaric acid is preferably 1:0.5. Compound A fumarate crystalline form I has one, more or all of the following characteristic XRPD peaks in terms of 2θ±0.2°: 4.703°, 5.234°, 7.100°, 9.515°, 10.561°, 10.864°, 11.566°, 14.336°, 15.324°, 15.926°, 16.625°, 17.834°, 18.671°, 19.193°, 19.904°, 20.972°, 21.320°, 21.607°, 21.908°, 22.058°, 23.118°, 23.487°, 24.089°, 24.899°, 25.162°, 25.337°, 26.463°, 26.767°, 28.444°, 28.809°, 29.017°, 29.496°, 30.621°, 31.059°, 32.281°, 32.640°, 33.214°, 33.407°, 34.009°, 34.480°, 35.672°, 37.870°, 38.210°, 39.076°, 39.488°.

One aspect, the fumarate crystalline form I has at least one characteristic XRPD peak in terms of 2θ selected from 9.5°±0.2°, 10.5°±0.2°, 14.3°±0.2° and 21.9°±0.2°; preferably, the fumarate crystalline form I has characteristic XRPD peaks in terms of 2θ: 9.5°±0.2°, 10.5°±0.2°, 14.3°±0.2° and 21.9°±0.2°.

Another aspect, the fumarate crystalline form I has at least one characteristic XRPD peak in terms of 2θ selected from 15.9°±0.2°, 16.6°±0.2°, 19.1°±0.2° and 22.0°±0.2°; preferably, the fumarate crystalline form I has characteristic XRPD peaks in terms of 2θ: 15.9°±0.2°, 16.6°±0.2°, 19.1°±0.2° and 22.0°±0.2°.

Another aspect, the fumarate crystalline form I has at least one characteristic XRPD peak in terms of 2θ selected from 4.7°±0.2°, 10.8°±0.2°, 19.9°±0.2°, 21.3°±0.2° and 26.7°±0.2°; preferably, the fumarate crystalline form I has characteristic XRPD peaks in terms of 2θ: 4.7°±0.2°, 10.8°±0.2°, 19.9°±0.2°, 21.3°±0.2° and 26.7°±0.2°.

Another aspect, the fumarate crystalline form I has at least one characteristic XRPD peak in terms of 2θ selected from 9.5°±0.2°, 10.5°±0.2°, 14.3°±0.2°, 21.9°±0.2°, 15.9°±0.2°, 16.6°±0.2°, 19.1°±0.2°, 22.0°±0.2°, 4.7°±0.2° 10.8°±0.2°, 19.9°±0.2°, 21.3°±0.2° and 26.7°±0.2°; preferably, the fumarate crystalline form I has characteristic XRPD peaks in terms of 2θ: 9.5°±0.2°, 10.5°±0.2°, 14.3°±0.2°, 21.9°±0.2°, 15.9°±0.2° 16.6°±0.2°, 19.1°±0.2°, 22.0°±0.2°, 4.7°±0.2°, 10.8°±0.2°, 19.9°±0.2°, 21.3°±0.2° and 26.7°±0.2°.

Another aspect, the fumarate crystalline form I has an XRPD pattern substantially as shown in FIG. 24.

The present invention also provides a preparation method of the above compound A fumarate crystalline form I, comprising the following steps:
contacting compound A with fumaric acid in a solvent to form compound A fumarate;
precipitating compound A fumarate as compound A fumarate form I.

In the above method, the solvent is isopropanol.

In the above method, the solvent is butanone.

In the above method, the solvent is 2-methyltetrahydrofuran.

In the above method, the solvent is acetonitrile/water, and the ratio of acetonitrile to water is preferably ranging from 15:1 to 25:1, such as 16:1, 17:1, 18:1, 19:1, 20:1, 21:1, 22:1, 23:1, 24:1.

In the above method, the precipitation is carried out at room temperature.

Compared with other salts of compound A and crystalline forms thereof, compound A fumarate and crystalline form I thereof have more advantages of acid-base ratio rationality in salt formation, single crystalline form under various crystallization systems, crystallinity, no solvation, stability (including solid storage stability, mechanical grinding stability and tableting stability), hygroscopicity, no polymorphism and the like, which means that they have a compromise of beneficial properties in these respects, so that the production and storage processes of related medicaments of the compound A can be simplified in operation and reduced in requirements, for example, impurities can be conveniently removed, the influence of fluctuation of production conditions is reduced, and the requirement on storage conditions is reduced.

A fourth aspect of the present invention provides a pharmaceutical composition, which comprises the above salt or the crystalline form, and a pharmaceutically acceptable carrier.

### BRIEF DESCRIPTION OF THE DRAWINGS

FIG. 1 depicts a polarization microscope photograph of free base crystalline form I (batch No.: A16803-008S3).

FIG. 2 depicts an XRPD pattern of starting material free base crystalline form I (batch No.: A16803-008S3).

FIG. 3 depicts a DSC and TGA diagram of free base crystalline form I (batch No.: A16803-008S3).

FIG. 4 depicts a DVS weight change curve profile of free base crystalline form I (batch No.: A16803-008S3).

FIG. 5 depicts a DVS isothermal adsorption/desorption curve profile of free base crystalline form I (batch No.: A16803-008S3).

FIG. 6 depicts an XRPD pattern of free base crystalline form I before and after DVS analysis.

FIG. 7 depicts an XRPD overlay of free base.

FIG. 8 depicts a DSC and TGA diagram of free base crystalline form II (batch No.: A16498-004A).

FIG. 9 depicts a DSC and TGA diagram of free base crystalline form III (batch No.: A16498-004C).

FIG. 10 depicts a DSC and TGA diagram of free base crystalline form IV (batch No.: A16498-004D).

FIG. 11 depicts an XRPD overlay pattern of hydrochloride.

FIG. 12 depicts an XRPD overlay pattern of sulfate.

FIG. 13 depicts an XRPD overlay pattern of p-toluenesulfonate.

FIG. 14 depicts an XRPD overlay pattern of mesylate.

FIG. 15 depicts an XRPD overlay pattern of besylate.

FIG. 16 depicts an XRPD overlay pattern of oxalate.

FIG. 17 depicts an XRPD overlay pattern of maleate.

FIG. 18 depicts an XRPD overlay pattern of phosphate.

FIG. 19 depicts an XRPD overlay pattern of phosphate crystalline form I (batch No.: A16498-045A1).

FIG. 20 depicts an XRPD overlay pattern of L-camphor sulfonate.

FIG. 21 depicts an XRPD overlay pattern of gentianate.

FIG. 22 depicts an XRPD overlay pattern of tartrate.

FIG. 23 depicts an XRPD overlay pattern of fumarate.

FIG. 24 depicts an XRPD overlay pattern of fumarate crystalline form I (batch No.: A16498-045C1).

FIG. 25 depicts a PLM pattern of fumarate crystalline form I (batch No.: A16498-067-B1).

FIG. 26 depicts an XRPD pattern of fumarate crystalline form I (batch No.: A16498-067-B1).

FIG. 27 depicts a DSC and TGA diagram of fumarate crystalline form I (batch No.: A16498-067-B1).

FIG. 28 depicts a ¹H-NMR spectrum of fumarate crystalline form I (batch No.: A16498-067-B1).

FIG. 29 depicts an XRPD overlay pattern of crystalline salts (I/II).

FIG. 30 depicts an XRPD overlay pattern of crystalline salts (II/II).

FIG. 31 depicts an XRPD overlay pattern of anhydrous crystalline form before and after stability study.

FIG. 32 depicts an XRPD overlay pattern of mesylate crystalline form I before and after stability study.

FIG. 33 depicts a polarization microscope photograph of phosphate crystalline form I (batch No.: A16498-045A1).

FIG. 34 depicts a DSC and TGA diagram of phosphate crystalline form I (batch No.: A16498-045A1).

FIG. 35 depicts a DVS weight change curve profile of phosphate crystalline form I (batch No.: A16498-045A1).

FIG. 36 depicts a DVS sorption curve profile of phosphate crystalline form I (batch No.: A16498-045A1).

FIG. 37 depicts an XRPD pattern of phosphate crystalline form I (batch No.: A16498-045A1) before and after DVS analysis.

FIG. 38 depicts a polarization microscope photograph of fumarate crystalline form I (batch No.: A16498-045C1).

FIG. 39 depicts a DSC and TGA diagram of fumarate crystalline form I (batch No.: A16498-045C1).

FIG. 40 depicts a DVS weight change curve profile of fumarate crystalline form I (batch No.: A16498-045C1).

FIG. 41 depicts a DVS sorption curve profile of fumarate crystalline form I (batch No.: A16498-045C1).

FIG. 42 depicts an XRPD pattern of fumarate crystalline form I (batch No.: A16498-045C1) before and after DVS analysis.

FIG. 43 depicts a biological solvent solubility data plot of free base, phosphate and fumarate.

FIG. 44 depicts an XRPD overlay pattern of residual solid of free base crystalline form I in water.

FIG. 45 depicts an XRPD overlay pattern of residual solid of free base crystalline form I in SGF.

FIG. 46 depicts an XRPD overlay pattern of residual solid of free base crystalline form I in FaSSIF.

FIG. 47 depicts an XRPD overlay pattern of residual solid of free base crystalline form I in FeSSIF.

FIG. 48 depicts an XRPD overlay pattern of residual solid of phosphate crystalline form I in water.

FIG. 49 depicts an XRPD overlay pattern of residual solid of phosphate crystalline form I in SGF.

FIG. 50 depicts an XRPD overlay pattern of residual solid of phosphate crystalline form I in FaSSIF.

FIG. 51 depicts a DSC and TGA diagram of phosphate form 3.

FIG. 52 depicts an XRPD overlay pattern of residual solid of phosphate crystalline form I in FeSSIF.

FIG. 53 depicts an XRPD overlay pattern of residual solid of fumarate crystalline form I in water.

FIG. 54 depicts an XRPD overlay pattern of residual solid of fumarate crystalline form I in SGF.

FIG. 55 depicts an XRPD overlay pattern of residual solid of fumarate crystalline form I in FaSSIF.

FIG. 56 depicts an XRPD overlay pattern of residual solid of fumarate crystalline form I in FeSSIF.

FIG. 57 depicts an XRPD overlay pattern of free base crystalline form I before and after stability analysis.

FIG. 58 depicts an HPLC overlay pattern of free base crystalline form I before and after stability analysis.

FIG. 59 depicts an XRPD overlay pattern of phosphate crystalline form I before and after stability analysis.

FIG. 60 depicts an HPLC overlay pattern of phosphate crystalline form I before and after stability analysis.

FIG. 61 depicts an XRPD overlay pattern of fumarate crystalline form I before and after stability analysis.

FIG. 62 depicts an HPLC overlay pattern of fumarate crystalline form I before and after stability analysis.

FIG. 63 depicts a DVS weight change curve profile of fumarate crystalline form I.

FIG. 64 depicts a DVS sorption curve profile of fumarate crystalline form I.

FIG. 65 depicts an XRPD overlay pattern of fumarate crystalline form I before and after grinding.

FIG. 66 depicts an XRPD overlay pattern of fumarate crystalline form I before and after tableting.

FIG. 67 depicts a histogram of roughly measured solubility of fumarate crystalline form I.

FIG. 68 depicts XRPD overlay patterns of samples crystallized by evaporation.

FIG. 69 depicts XRPD overlay patterns of samples crystallized by cooling.

FIG. 70 depicts XRPD overlay patterns of samples crystallized by antisolvent precipitation.

FIG. 71 depicts XRPD overlay patterns of samples triturated for 3 days at room temperature.

FIG. 72 depicts XRPD overlay patterns of samples triturated for 3 days at 50° C.

FIG. 73 depicts XRPD overlay patterns of samples triturated for 7 days at room temperature.

FIG. 74 depicts XRPD overlay patterns of samples triturated for 7 days at 50° C.

### ABBREVIATIONS

### Chemical Reagent Abbreviations

ACN acetonitrile
n-BuOH n-butanol
DMSO dimethyl sulfoxide
DCM dichloromethane
EtOAc ethyl acetate
EtOH ethanol
Hept n-heptane
IPA isopropanol
MeOH methanol
2-Me THF 2-methyltetrahydrofuran
MEK butanone
MTBE methyl tert-butyl ether
MIBK methyl isobutyl ketone
THF tetrahydrofuran
IPAc isopropyl acetate
Tol toluene

### Other Abbreviations (Sorted Alphabetically)

Conc. concentration
DSC differential scanning calorimeter
DVS dynamic vapor sorption
eq. equivalent
FaSSIF fasting state simulated intestinal fluid
FB free base
FeSSIF feeding state simulated intestinal fluid
h hour
HPLC high performance liquid chromatography
¹H-NMR hydrogen nuclear magnetic resonance
LOD limit of detection
LOQ limit of quantitation
IC ion chromatography
PLM polarizing microscope
RT room temperature (20-25° C.)
SGF simulated gastric fluid
SM starting material
TGA thermogravimetry
XRPD X-ray power diffraction

### DETAILED DESCRIPTION

### 1. Characterization of Starting Materials

### 1.1 Characterization of Solid-State Properties

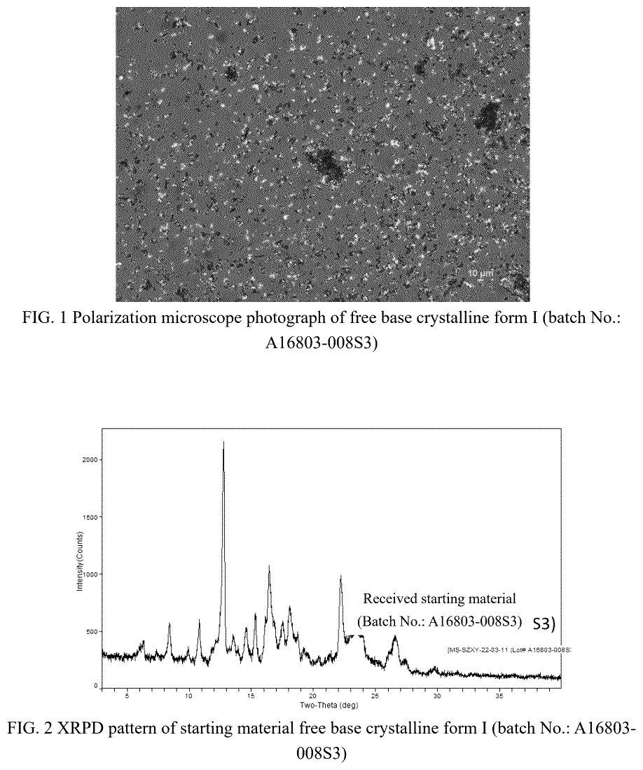

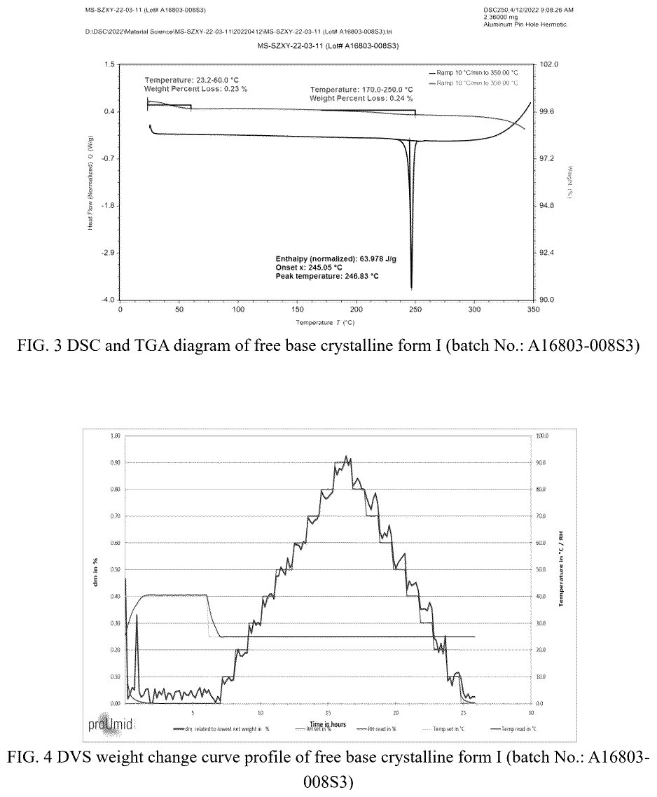

Free base of compound A (batch No.: A16803-008S3) was fully characterized as a starting material. The characterization data were shown in FIGS. 1-3.

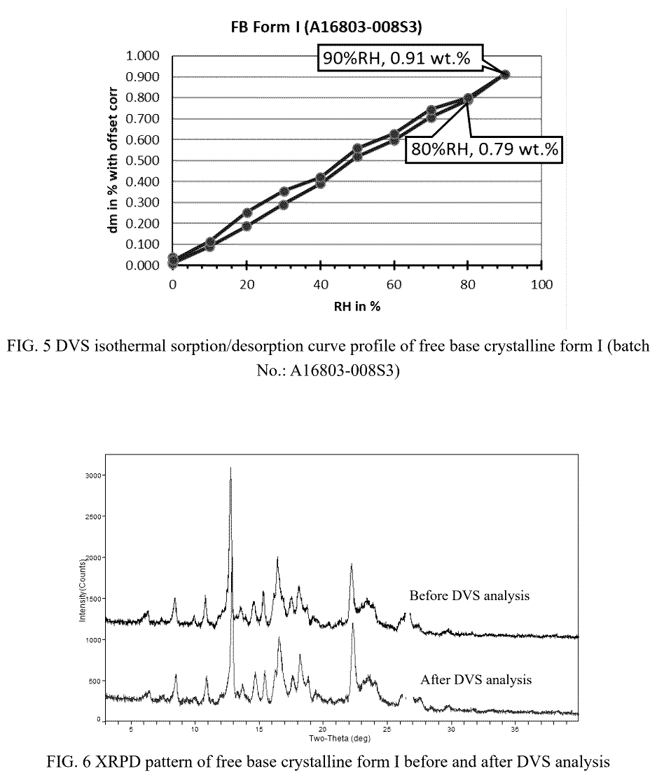

The starting material presented small crystal with agglomeration, the crystallinity was mean level degree, named as free base crystalline form I. TGA showed that the sample had a 0.2% weight loss during heating from room temperature to 60° C. and a 0.2% weight loss from 170° C. to 250° C., speculating that the sample has been dehydrated and desolvated. There was only one endothermic peak at 245° C. in the DSC diagram, which was caused by melting. Therefore, the free base crystalline form I was an anhydrous form. As shown in FIGS. 4-6, the DVS results showed that free base crystalline form I was slightly hygroscopic, the vapor sorption gains were about 0.8% and 0.9% under the condition of 80% RH and 90% RH respectively, and the crystalline form remained unchanged after DVS analysis.

### 1.2 Roughly Measured Solubility

The rough solubility test of free base of compound A was visually evaluated by solvent addition, and the results were summarized in Table 1.

This batch of material was soluble (>50 mg/mL) in butanone, tetrahydrofuran or 2-methyltetrahydrofuran, but insoluble (<1 mg/mL) in water, toluene or n-heptane. During the experiment, solid precipitation was observed in the isopropanol solution and the butanone solution, indicating that new crystalline forms may form.

TABLE 1
Rough solubility of starting material (batch No .: A16803-008S3)

| | | Solubility | | | Solubility |
|---|---|---|---|---|---|
| No. | Solvent | (mg/mL) | No. | Solvent (v/v) | (mg/mL) |
| 1 | methanol | 3-5 | 10 | 2-methyltetra | 50-100 |
| | | | | hydrofuran | |
| 2 | ethanol | 5-20 | 11 | methyl tert-butyl | 2-5 |
| | | | | ether | |
| 3 | isopropanol | 20-50 | 12 | toluene | <1 |
| 4 | acetone | 20-50 | 13 | n-heptane | <1 |
| 5 | butanone | 50-100 | 14 | dichloromethane | 3-5 |
| 6 | methyl isobutyl | 5-20 | 15 | acetonitrile | 1-5 |
| | ketone | | | | |
| 7 | ethyl acetate | 5-20 | 16 | water | <1 |
| 8 | isopropyl acetate | 5-20 | 17 | acetonitrile/water, | 3-5 |
| | | | | 19/1 | |
| 9 | tetrahydrofuran | >100 | / | / | / |

### 1.3 Research on Trituration of Free Base (Blank Experiment of Salt Formation)

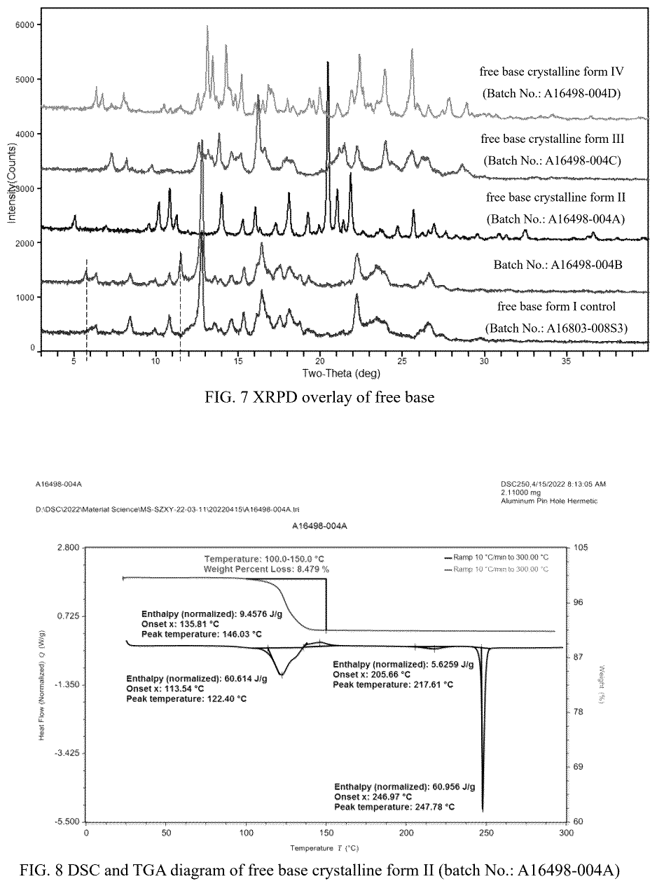

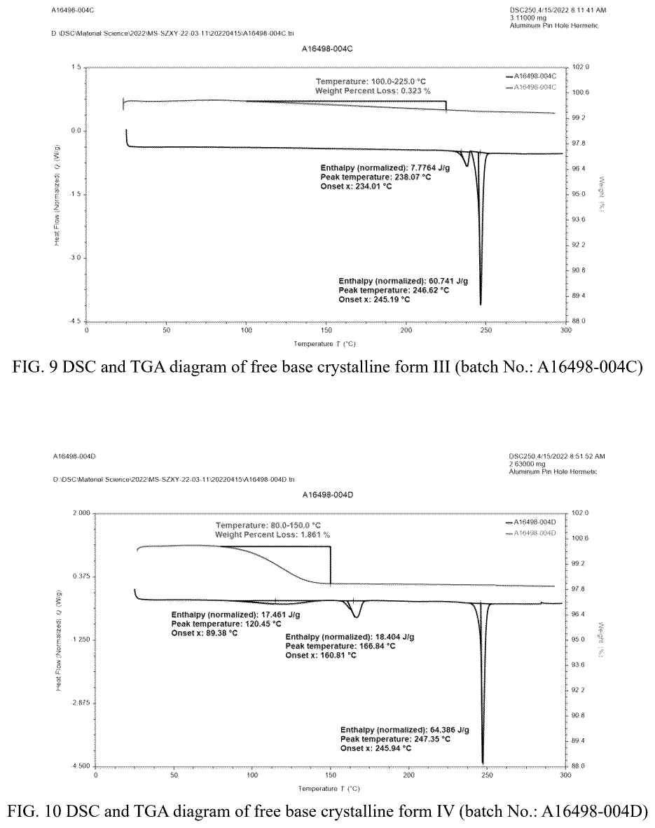

As shown in Table 2, three new crystalline forms of free base were determined through blank experiments, including free base crystalline form II, free base crystalline form III, and free base crystalline form IV, respectively. The XRPD overlay pattern was shown in FIG. 7, and the DSC and TGA diagrams were shown in FIGS. 8-10.

The XRPD pattern showed that free base crystalline form II has high crystallinity. 8.6 wt. % Residual of isopropanol was detected by ¹H-NMR. TGA showed that the sample has an 8.5% weight loss in the temperature range of 100-150° C., attributed to desolventization. Multiple endothermic peaks were observed on the DSC curve. Therefore, it was determined that free base crystalline form II is an isopropanol solvate.

The XRPD pattern showed that free base crystalline form III has moderate crystallinity. Two overlapping endothermic peaks were detected by DSC, it was determined to be caused by sample melting. 0.2 wt. % Residual of 2-methyltetrahydrofuran was detected by ¹H-NMR, and TGA showed that a 0.3% weight loss at the range of 100-225° C., suggesting that 2-methyltetrahydrofuran was removed. Based on the characterization data, it is determined that free base crystalline form III is an anhydrous form.

Free base crystalline form IV was obtained from raw material by triturating in a mixed solvent of acetonitrile/water, it has moderately higher crystallinity. 2.2 wt. % Residual of acetonitrile was detected by ¹H-NMR, three endothermic peaks were detected by DSC, and TGA diagram showed a 1.9% weight loss of the sample at the range of 80-150° C., attributed to the removal of acetonitrile. Therefore, it is determined that the free base crystalline form IV is an acetonitrile solvate.

The characterization data of various crystalline forms of free base were summarized in Table 3. Because of higher melting point and enthalpy value, free base crystalline form I was selected and further researched.

TABLE 2
Preparation of new crystalline forms of free base

| Batch | | | |
|---|---|---|---|
| No .: | | | |
| A16498- | Solvent | | XRPD |
| 004 | (v/v) | Experimental procedure | results |
| A | isopropanol | At the beginning, a solution with a | free base |
| | | concentration of 40 mg/mL was obtained, and | crystalline |
| | | after stirring for about 2 minutes, solids | form II |
| | | precipitated. After stirring for 20 hours, it | |
| | | remained a suspension. | |
| B | butanone | At the beginning, a solution with a | free base |
| | | concentration of 50 mg/mL was obtained, and | crystalline |
| | | after stirring for 20 hours, it was kept as such. | form I + |
| | | 1 mL of n-heptane was added at room | 5.7º + 11.5° |
| | | temperature, oil precipitated, and it was stirred | (2θ) |
| | | at room temperature for 7 hours, and then | |
| | | turned into a suspension. | |
| C | 2- | At the beginning, a solution with a | free base |
| | methyltetrahydrofuran | concentration of 60 mg/mL was obtained, and | crystalline |
| | | after stirring for 20 hours, it was kept as such. | form III |
| | | 1 mL of n-heptane was added at room | |
| | | temperature, oil precipitated, and it was stirred | |
| | | at room temperature for 7 hours, and then | |
| | | turned into a suspension. | |
| D | acetonitrile/water, | At the beginning, a suspension solution with a | free base |
| | 19/1 | concentration of 50 mg/mL was obtained, and | crystalline |
| | | after stirring for 20 hours, it was kept as such. | form IV |

TABLE 3
Summary of free base crystalline forms data

| | | | DSC | | | |
|---|---|---|---|---|---|---|
| | | | Melting | | | |
| | | | point/peak | TGA | | |
| | | | (endothermic | Weight | Residual | |
| Free base | | | peak ° C.), | loss %/ | of solvent | |
| crystalline | Crystalline | Crystallinity | Enthalpy value | temperature | ¹H-NMR | |
| form | form | (XRPD) | (J/g) | (° C.) | (wt. %) | Notes |
| crystalline | anhydrous | moderate | 245/247, 64 | 0.2/170-250 | 0.2 methyl | received |
| form I | crystalline | | | | tert-butyl | starting |
| | form | | | | ether | material |
| crystalline | isopropanol | high | multiple | 8.5/100-150 | 8.6 | / |
| form II | solvates | | endothermic | | isopropanol | |
| | | | peaks | | | |
| crystalline | anhydrous | moderate | 234/238, 8 | 0.3/100-225 | 0.2 | / |
| form III | crystalline | | 245/247, 61 | | 2-methyltetra- | |
| | form | | | | hydrofuran | |
| crystalline | acetonitrile | moderately | 89/120, 17 | 1.9/80-150 | 2.2 | / |
| form IV | solvates | higher | 161/167, 18 | | acetonitrile | |
| | | | 246/247, 64 | | | |

### 2. Preparation of a Pharmaceutically Acceptable Salt of Compound A

### 2.1 Preparation of Hydrochloride

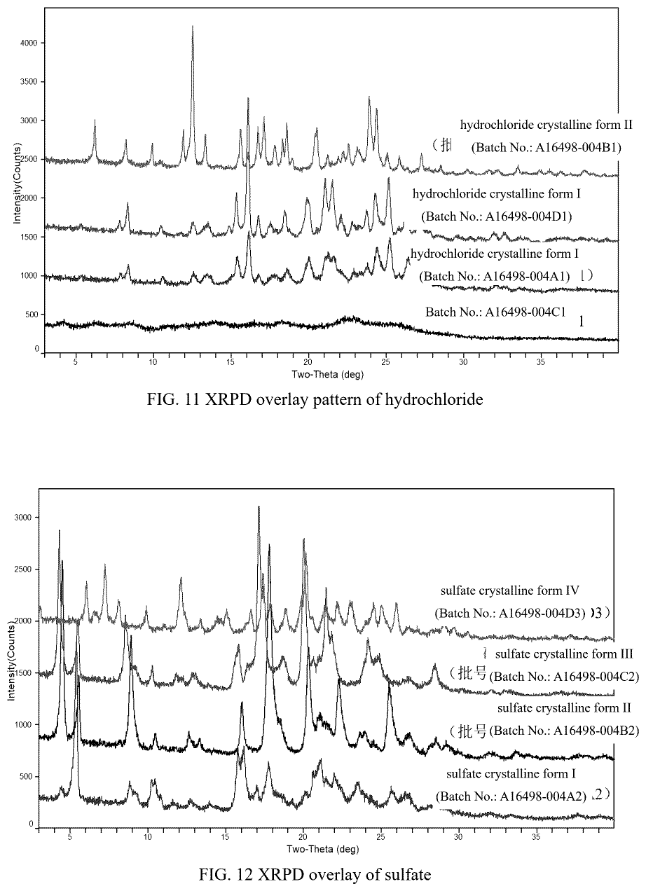

Two crystalline forms of hydrochloride were obtained by salt formation of free base and hydrochloric acid, named hydrochloride crystalline form I and hydrochloride crystalline form II respectively (the results were shown in Table 4). The XRPD pattern was shown in FIG. 11.

TABLE 4
Preparation of hydrochloride

| Batch | | | |
|---|---|---|---|
| No. | | | |
| A16498- | Solvent | | XPRD |
| 004 | (v/v) | Experimental procedure | Results |
| A1 | isopropanol | After stirring at room temperature for 2 | hydrochloride |
| | | hours, the solution turned into a | crystalline |
| | | suspension, and it was kept as such after | form I |
| | | stirring for 18 hours. | |
| B1 | butanone | At the beginning, a gel was obtained, | hydrochloride |
| | | after stirring at room temperature for 2 | crystalline |
| | | hours, which turned into a thick | form II |
| | | suspension, then 0.2 mL of butanone | |
| | | was added and after 18 hours of stirring, | |
| | | a suspension was finally obtained. | |
| C1 | 2- | At the beginning, a gel was obtained, | amorphous |
| | methyltetrahydrofuran | after stirring at room temperature for 20 | |
| | | hours, which turned into a suspension. | |
| D1 | acetonitrile/water, | At the beginning, a gel was obtained, | hydrochloride |
| | 19/1 | after stirring at room temperature for 20 | crystalline |
| | | hours, which turned into a suspension. | form I |

### 2.1.1 Hydrochloride Crystalline Form I

Hydrochloride crystalline form I can be obtained in two solvent systems: isopropanol and acetonitrile/water (19/1, v/v).

Hydrochloride crystalline form I (batch No.: A16498-004A1): about 25 mg of starting material free base (batch No.: A16803-008S3) was weighed, 0.5 mL of isopropanol was added at room temperature (~24° C.) and the mixture was stirred for about 10 minutes to obtain a white suspension. 42 μL of concentrated hydrochloric acid was added into 0.5 mL of isopropanol, mixing well, and 55 μL of the mixture (containing 1.1 equivalents of hydrochloric acid) was added to a free base suspension to obtain a clear solution. After stirring at room temperature for 2 hours, the solution turned to a suspension, and after stirring for 18 hours, it remained a suspension. The suspension was filtered by using a syringe equipped with 0.22 μm microporous filter membrane to obtain a solid sample, and the solid was dried under vacuum at 40° C. for 5 hours to obtain the sample. It was identified as hydrochloride crystalline form I by X-ray powder diffraction.

Hydrochloride crystalline form I (batch No.: A16498-004D1): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. 42 μL of concentrated hydrochloric acid was added into 0.5 mL of isopropanol, mixing well, and 55 μL of the mixture (containing 1.1 equivalents of hydrochloric acid) was added to a free base suspension to obtain a gel. The gel was stirred at room temperature for 20 hours to turned to a suspension, the suspension was filtered by using a syringe equipped with 0.22 μm microporous filter membrane to obtain a solid sample, and the solid was dried under vacuum at 40° C. for 5 hours to obtain the sample. It was identified as hydrochloride crystalline form I by X-ray powder diffraction.

### 2.1.2 Hydrochloride Crystalline Form II

Hydrochloride crystalline form II (batch No.: A16498-004B1): about 25 mg of starting material free base (batch No.: A16803-008S3) was weighed, 0.5 mL of butanone was added at room temperature (24° C.) and the mixture was stirred for about 10 minutes to obtain a clear solution. 42 μL of concentrated hydrochloric acid was added to 0.5 mL of butanone, mixing well, and then 55 μL of the mixture (containing 1.1 equivalents of hydrochloric acid) was added to a free base suspension to obtain a gel, after stirring at room temperature for 2 hours, which turned into a thick suspension, then 0.2 mL of butanone was added and after 18 hours of stirring, a suspension was finally obtained. The suspension was filtered by using a syringe equipped with 0.22 m microporous filter membrane to obtain a solid sample, and the solid was dried under vacuum at 40° C. for 5 hours to obtain the sample. It was identified as hydrochloride crystalline form II by X-ray powder diffraction.

### 2.2 Sulfate Crystalline Form I, Crystalline Form 20, Crystalline Form III, Crystalline Form IV

Four sulfate crystalline forms (crystalline form I, crystalline form II, crystalline form III, and crystalline form IV) were obtained by salt formation of 1.1 equivalents and 0.55 equivalents of sulfuric acid with free base sample (the results were shown in Tables 5 and 6). The XRPD pattern was shown in FIG. 12.

TABLE 5
Preparation of sulfate (1.1 equivalents of sulfuric acid)

| Batch | | | |
|---|---|---|---|
| No. | | | |
| A16498- | Solvent | | XPRD |
| 004 | (v/v) | Experimental procedure | Results |
| A2 | isopropanol | At the beginning, a solution was | sulfate |
| | | obtained, and was kept as such | crystalline |
| | | after stirring at room temperature | form I |
| | | for 2 hours, and after | |
| | | stirring for 20 hours, the | |
| | | suspension was kept as such. | |
| | | After adding n-heptane (1 mL), | |
| | | solid precipitates were observed. | |
| | | After stirring for 18 hours, a | |
| | | suspension was obtained. | |
| B2 | butanone | At the beginning, a solution | sulfate |
| | | was obtained, a suspension | crystalline |
| | | was obtained after stirring | form II |
| | | for 2 hours, and after stirring for | |
| | | 18 hours, it remained | |
| | | a suspension. | |
| C2 | 2- | At the beginning, a solution was | sulfate |
| | methyl- | obtained, a suspension | crystalline |
| | tetrahydrofuran | was obtained after stirring | form III |
| | | for 2 hours, and after | |
| | | stirring for 18 hours, it | |
| | | remained a suspension. | |
| D2 | acetonitrile/ | At the beginning, the solution | low degree |
| | water, | was almost clear, and after | of |
| | 19/1 | stirring for 2 hours, a thick | crystallinity |
| | | suspension was obtained. | |
| | | After adding 0.2 mL of solution, | |
| | | after stirring for 18 hours, | |
| | | a suspension was obtained. | |

TABLE 6
Preparation of sulfate (0.55 eq. of sulfuric acid)

| Batch | | | |
|---|---|---|---|
| No. | | | |
| A16498- | Solvent | | XPRD |
| 004 | (v/v) | Experimental procedure | Results |
| A3 | isopropanol | At the beginning, a solution | low |
| | | was obtained, and after | degree of |
| | | stirring for 2 hours, a | crystallinity |
| | | thick suspension was obtained. | |
| | | After adding 0.2 mL of | |
| | | solution, after stirring | |
| | | for 18 hours, a suspension | |
| | | was obtained. | |
| B3 | butanone | At the beginning, a solution | low |
| | | was obtained, a suspension | degree of |
| | | was obtained after stirring | crystallinity |
| | | for 2 hours, and after | |
| | | stirring for 18 hours, it | |
| | | remained a suspension. | |
| C3 | 2- | At the beginning, a | approximate |
| | methyl- | solution was obtained, and | amorphous |
| | tetrahydrofuran | after stirring for 2 hours, it | |
| | | became almost clear. After | |
| | | stirring for 18 hours, a | |
| | | suspension was finally | |
| | | obtained. | |
| D3 | acetonitrile/ | At the beginning, an oil | sulfate |
| | water, | precipitated and which was | crystalline |
| | 19/1 | kept as such after stirring for | form IV |
| | | 20 hours. After it was stirred | |
| | | at 50° C. for 7 hours, a | |
| | | suspension was obtained. | |

### 2.2.1 Sulfate Crystalline Form I

Sulfate crystalline form I (batch No.: A16498-004A2): about 25 mg of starting material free base (batch No.: A16803-008S3) was weighed, 0.5 mL of isopropanol was added at room temperature (~24° C.) and the mixture was stirred for about 10 minutes to obtain a white suspension. 28 μL of concentrated sulfuric acid was added to 0.5 mL of isopropanol, mixing well, and then 53 μL of the mixture (containing 1.1 equivalents of sulfuric acid) was added to a free base suspension to obtain a clear solution. After stirring at room temperature for 22 hours, it remained a solution, solid precipitates were observed by adding 1 mL of n-heptane, after stirring for 18 hours, a suspension was obtained. The suspension was filtered by using a syringe equipped with 0.22 μm microporous filter membrane to obtain a solid sample, and the solid was dried under vacuum at 40° C. for 5 hours to obtain the sample. It was identified as sulfate crystalline form I by X-ray powder diffraction.

### 2.2.2 Sulfate Crystalline Form II

Sulfate crystalline form II (batch No.: A16498-004B2): about 25 mg of starting material free base (batch No.: A16803-008S3) was weighed, 0.5 mL of butanone at was added room temperature (~24° C.) and the mixture was stirred for about 10 minutes to obtain a clear solution. 28 μL of concentrated sulfuric acid was added to 0.5 mL of isopropanol, mixing well, and then 53 μL of the mixture (containing 1.1 equivalents of sulfate acid) was added to a free base solution to obtain a solution, after stirring for 2 hours, which turned into a suspension, after continuing to stir for 18 hours, which remained a suspension. The suspension was filtered by using a syringe equipped with 0.22 μm microporous filter membrane to obtain a solid sample, and the solid was dried under vacuum at 40° C. for 5 hours to obtain the sample. It was identified as sulfate crystalline form II by X-ray powder diffraction.

### 2.2.3 Sulfate Crystalline Form III

Sulfate crystalline form III (batch No.: A16498-004C2): a solution of free base (batch No.: A16803-008S3) in 2-methyltetrahydrofuran with a concentration of 60 mg/mL was prepared, and 416 μL of the solution was taken into a glass vial. 28 μL of concentrated sulfuric acid was added to 0.5 mL of 2-methyltetrahydrofuran, mixing well, and then 53 μL of the mixture (containing 1.1 equivalents of sulfate acid) was added to a free base solution to obtain a solution, after stirring for 2 hours, which turned into a suspension, after stirring for 18 hours, which remained a suspension. The suspension was filtered by using a syringe equipped with 0.22 μm microporous filter membrane to obtain a solid sample, and the solid was dried under vacuum at 40° C. for 5 hours to obtain the sample. It was identified as sulfate crystalline form III by X-ray powder diffraction.

### 2.2.4 Sulfate Crystalline Form IV

Sulfate crystalline IV (batch No.: A16498-004D3): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. 28 μL of concentrated sulfuric acid was added to 0.5 mL of acetonitrile/water (19/1, v/v), mixing well, and then 27 μL of the mixture (containing 0.55 equivalents of sulfuric acid) was added to a free base suspension. At the beginning, an oil precipitated, which was kept as such after stirring for 20 hours. It was stirred at 50° C. for 7 hours, and turned to a suspension. The suspension was filtered by using a syringe equipped with 0.22 μm microporous filter membrane to obtain a solid sample, and the solid was dried under vacuum at 40° C. for 5 hours to obtain the sample. It was identified as sulfate crystalline form IV by X-ray powder diffraction.

2.3 p-Toluenesulfonate

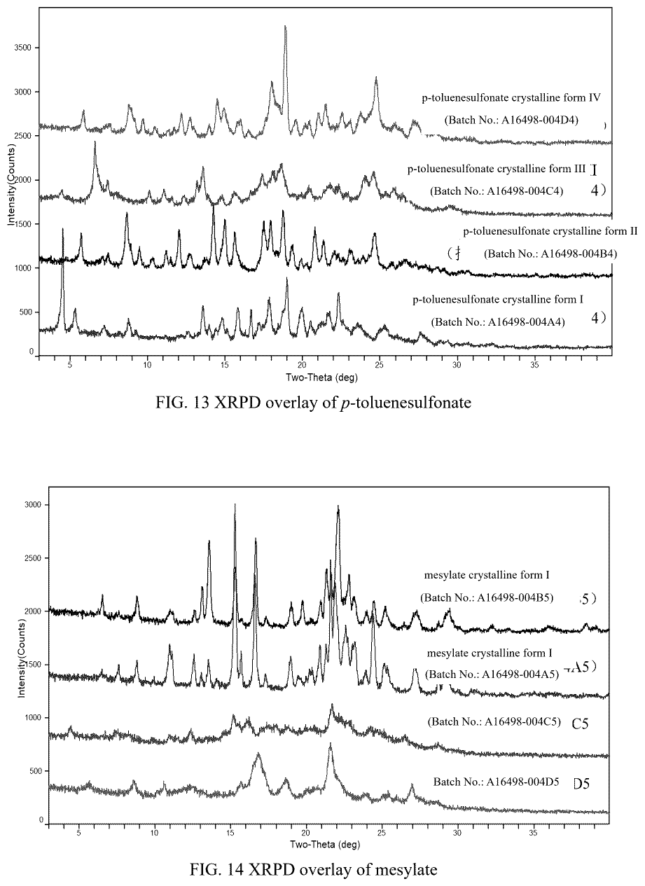

Four p-toluenesulfonate crystalline forms were obtained by salt formation of p-toluenesulfonic acid and free base sample, named p-toluenesulfonate crystalline form I, crystalline form II, crystalline form III and crystalline form IV respectively. (The results were shown in Table 7). The XRPD pattern was shown in FIG. 13.

TABLE 7
Preparation of p-toluenesulfonate

| Batch No. | | | |
|---|---|---|---|
| A16498- | Solvent | | |
| 004 | (v/v) | Experimental procedure | XPRD Results |
| A4 | isopropanol | After stirring at room | p- |
| | | temperature for 2 | toluenesulfonate |
| | | hours, the solution | crystalline |
| | | turned into a thick | form I |
| | | suspension, then | |
| | | 0.2 mL of isopropanol | |
| | | was added and after | |
| | | 18 hours of stirring, a | |
| | | suspension was obtained. | |
| B4 | butanone | At the beginning, a | p- |
| | | solution was obtained, | toluenesulfonate |
| | | solid precipitates | crystalline form |
| | | were observed after | II |
| | | stirring for 2 hours, | |
| | | and after stirring for 18 | |
| | | hours, it remained | |
| | | a suspension. | |
| C4 | 2- | At the beginning, a | p- |
| | methyl- | solution was obtained, | toluenesulfonate |
| | tetra- | solid precipitates | crystalline form |
| | hydrofuran | were observed after | III |
| | | stirring for 2 min, and | |
| | | after stirring for 20 | |
| | | hours, it remained | |
| | | a suspension. | |
| D4 | acetonitrile/ | After stirring at room | p- |
| | water, | temperature for 2 | toluenesulfonate |
| | 19/1 | hours, the solution | crystalline form |
| | | turned to a dilute | IV |
| | | suspension, and a | |
| | | suspension was obtained | |
| | | after stirring for 18 hours. | |

2.3.1 p-Toluenesulfonate Crystalline Form I

p-Toluenesulfonate crystalline form I (batch No.: A16498-004A4): about 25 mg of starting material free base (batch No.: A16803-008S3) was weighed, 0.5 mL of isopropanol was added at room temperature (~24° C.) and the mixture was stirred for about 10 minutes to obtain a white suspension. 8.6 mg (1.1 equivalents) of p-toluenesulfonic acid solid was added to obtain a clear solution. After stirring at room temperature for 2 hours, the solution turned to a thick suspension, and 0.5 mL of isopropanol was added, a suspension was obtained after stirring for 18 hours. The suspension was filtered by using a syringe equipped with 0.22 μm microporous filter membrane to obtain a solid sample, and the solid was dried under vacuum at 40° C. for 5 hours to obtain the sample. It was identified as p-toluenesulfonate crystalline form I by X-ray powder diffraction.

2.3.2 p-Toluenesulfonate Crystalline Form II

p-Toluenesulfonate crystalline form II (batch No.: A16498-004B4): about 25 mg of starting material free base (batch No.: A16803-008S3) was weighed, 0.5 mL of butanone was added at room temperature (~24° C.) and the mixture was stirred for about 10 minutes to obtain a clear solution. 8.6 mg (1.1 equivalents) of p-toluenesulfonic acid solid was weighed and added to a free base solution to obtain a solution. Solid precipitates were observed after stirring for 2 hours, and after stirring for 18 hours, it remained a suspension. The suspension was filtered by using a syringe equipped with 0.22 μm microporous filter membrane to obtain a solid sample, and the solid was dried under vacuum at 40° C. for 5 hours to obtain the sample. It was identified as p-toluenesulfonate crystalline form II by X-ray powder diffraction.

2.3.3 p-Toluenesulfonate Crystalline Form III

p-Toluenesulfonate crystalline form III (batch No.: A16498-004C4): a solution of free base (batch No.: A16803-008S3) in 2-methyltetrahydrofuran with a concentration of 60 mg/mL was prepared, and 416 μL of the solution was taken into a glass vial. 8.6 mg (1.1 equivalents) of p-toluenesulfonic acid solid was weighed and added to a free base solution to obtain a solution, solid precipitates were observed after stirring for 2 min, and after stirring for 20 hours, it remained a suspension. The suspension was filtered by using a syringe equipped with 0.22 m microporous filter membrane to obtain a solid sample, and the solid was dried under vacuum at 40° C. for 5 hours to obtain the sample. It was identified as p-toluenesulfonate crystalline form III by X-ray powder diffraction.

2.3.4 p-Toluenesulfonate Crystalline Form IV

p-Toluenesulfonate crystalline IV (batch No.: A16498-0041D4): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. 8.6 mg (1.1 equivalents) of p-toluenesulfonic acid solid was weighed and added to a free base suspension, after stirring at room temperature for 2 hours, the solution turned to a dilute suspension. The suspension was filtered by using a syringe equipped with 0.22 μm microporous filter membrane to obtain a solid sample, and the solid was dried under vacuum at 40° C. for 5 hours to obtain the sample. It was identified as p-toluenesulfonate crystalline form IV by X-ray powder diffraction.

### 2.4 Mesylate Crystalline Form I

A mesylate crystalline form was obtained by salt formation of free base as the raw material and 1.1 equivalents of methanesulfonic acid, named mesylate crystalline form I. (The results were shown in Table 8). The XRPD pattern was shown in FIG. 14.

TABLE 8
Preparation of mesylate

| Batch | | | |
|---|---|---|---|
| No. | | | |
| A16498- | Solvent | | XPRD |
| 004 | (v/v) | Experimental procedure | Results |
| A5 | isopropanol | After stirring 20 hours and the | mesylate |
| | | solution was kept as such. Adding | crystalline |
| | | 1mL of n-heptane, oil precipitated, | form I |
| | | and then which was stirred at | |
| | | 50° C. for 7 hours to obtain | |
| | | a suspension finally. | |
| B5 | butanone | At the beginning, a solution was | mesylate |
| | | obtained, solid precipitates were | crystalline |
| | | observed after stirring for 2 hours, | form I |
| | | and after stirring for 18 hours, it | |
| | | remained a suspension. | |
| C5 | 2- | At the beginning, a solution was | low |
| | methyltetra- | obtained, solid precipitates were | degree of |
| | hydrofuran | observed after stirring for 2 hours, | crystallinity |
| | | and after stirring for 18 hours, it | |
| | | remained a suspension. | |
| D5 | acetonitrile/ | At the beginning, oil precipitated | low |
| | water, 19/1 | and after stirring at room | degree of |
| | | temperature for 20 hours, it became | crystallinity |
| | | almost clear. Adding 1 mL of water | |
| | | and stirring for 7 hours, solid | |
| | | precipitation occurred. | |

Mesylate crystalline form I (batch No.: A16498-004A5): about 25 mg of starting material free base (batch No.: A16803-008S3) was weighed, 0.5 mL of isopropanol was added at room temperature (~24° C.) and the mixture was stirred for about 10 minutes to obtain a white suspension. 48 mg of methanesulfonic acid was weighed and added to 0.5 mL of isopropanol, after dissolution, 55 μL of the mixture (containing 1.1 equivalents of methanesulfonic acid) was added to a free base suspension. At the beginning, a solution was obtained, and after stirring for 20 hours, it became almost clear. 1 mL of n-heptane was added, oil precipitated, and then the mixture was stirred at 50° C. for 7 hours to obtain a suspension. The suspension was filtered by using a syringe equipped with 0.22 μm microporous filter membrane to obtain a solid sample, and the solid was dried under vacuum at 40° C. for 5 hours to obtain the sample. It was identified as mesylate crystalline form I by X-ray powder diffraction.

Mesylate crystalline form I (batch No.: A16498-004B5): about 25 mg of starting material free base (batch No.: A16803-008S3) was weighed, 0.5 mL of butanone was added at room temperature (~24° C.) and the mixture was stirred for about 10 minutes to obtain a clear solution. 48 mg of methanesulfonic acid was weighed and added to 0.5 mL of butanone, after dissolution, 55 μL of the mixture (containing 1.1 equivalents of methanesulfonic acid) was added to a free base suspension to obtain a solution. Solid precipitates were observed after stirring for 2 hours, and after stirring for 18 hours, it remained a suspension. The suspension was filtered by using a syringe equipped with 0.22 μm microporous filter membrane to obtain a solid sample, and the solid was dried under vacuum at 40° C. for 5 hours to obtain the sample. It was identified as mesylate crystalline form I by X-ray powder diffraction.

### 2.5 Besylate

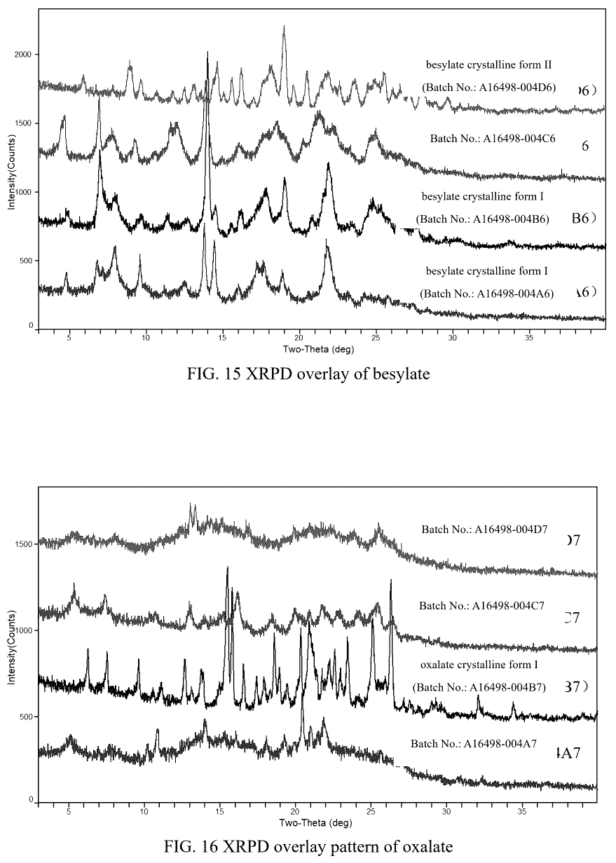

Two crystalline forms of besylate were obtained by salt formation of free base and benzenesulfonic acid, named besylate crystalline form I and besylate crystalline form II respectively. (The results are shown in Table 9). The XRPD pattern was shown in FIG. 15.

TABLE 9
Preparation of besylate

| Batch | | | |
|---|---|---|---|
| No. | | | |
| A16498- | Solvent | | XPRD |
| 004 | (v/v) | Experimental procedure | Results |
| A6 | isopropanol | After stirring for 2 hours, | besylate |
| | | the solution was kept | crystalline |
| | | as such and after stirring | form I |
| | | for 18 hours, it turned | |
| | | to a suspension. | |
| B6 | butanone | At the beginning, a solution | besylate |
| | | was obtained, solid | crystalline |
| | | precipitates were observed | form I |
| | | after stirring for 2 | |
| | | hours, and after stirring | |
| | | for 18 hours, it | |
| | | remained a suspension. | |
| C6 | 2- | At the beginning, a solution | similar to |
| | methyl- | was obtained, solid | besylate |
| | tetrahydrofuran | precipitates were observed | crystalline |
| | | after stirring for 2 | form I |
| | | hours, and after | |
| | | stirring for 18 hours, it | |
| | | remained a suspension. | |
| D6 | acetonitrile/ | At the beginning, the | besylate |
| | water, 19/1 | solution was almost clear, | crystalline |
| | | and after stirring at room | form II |
| | | temperature for 2 | |
| | | hours, it was turned to a | |
| | | thick suspension. After adding | |
| | | 0.2 mL of acetonitrile/water, | |
| | | after stirring for 18 hours, | |
| | | a suspension was obtained. | |

### 2.5.1 Besylate Crystalline Form I

Besylate crystalline form I can be obtained in two organic solvent systems: isopropanol and butanone.

Besylate crystalline form I (batch No.: A16498-004A6): about 25 mg of starting material free base (batch No.: A16803-008S3) was weighed, 0.5 mL of isopropanol was added at room temperature (~24° C.) and the mixture was stirred for about 10 minutes to obtain a white suspension. 9.2 mg (1.1 equivalents) of benzenesulfonic acid solid was weighed and added to a free base suspension to obtain a clear solution. After stirring for 2 hours, the solution was kept as such and after stirring for 18 hours, it turned to a suspension. The suspension was filtered by using a syringe equipped with 0.22 μm microporous filter membrane to obtain a solid sample, and the solid was dried under vacuum at 40° C. for 5 hours to obtain the sample. It was identified as besylate crystalline form I by X-ray powder diffraction.

Besylate crystalline form I (batch No.: A16498-004B6): about 25 mg of starting material free base (batch No.: A16803-008S3) was weighed, 0.5 mL of butanone was added at room temperature (~24° C.) and the mixture was stirred for about 10 minutes to obtain a clear solution. 9.2 mg (1.1 equivalents) of besylate acid solid was weighed and added to a free base solution to obtain a solution. Solid precipitates were observed after stirring for 2 hours, and after stirring for 18 hours, it remained a suspension. The suspension was filtered by using a syringe equipped with 0.22 μm microporous filter membrane to obtain a solid sample, and the solid was dried under vacuum at 40° C. for 5 hours to obtain the sample. It was identified as besylate crystalline form I by X-ray powder diffraction.

### 2.5.2 Besylate Crystalline Form II

Besylate crystalline II (batch No.: A16498-004D6): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. 9.2 mg (1.1 equivalents) of benzenesulfonic acid solid was weighed and added to a free base suspension to obtain an almost clear solution. After stirring at room temperature for 2 hours, it was turned to a thick suspension. After adding 0.2 mL of acetonitrile/water (19/1, v/v), and stirring for 18 hours, a suspension was obtained. The suspension was filtered by using a syringe equipped with 0.22 μm microporous filter membrane to obtain a solid sample, and the solid was dried under vacuum at 40° C. for 5 hours to obtain the sample. It was identified as besylate crystalline form II by X-ray powder diffraction.

### 2.6 Oxalate Crystalline Form I

One oxalate crystalline form was obtained by salt formation of free base and oxalic acid. (The results were shown in Table 10). The XRPD pattern was shown in FIG. 16.

TABLE 10
Preparation of oxalate

| Batch | | | |
|---|---|---|---|
| No. | | | |
| A16498- | Solvent | | XPRD |
| 004 | (v/v) | Experimental procedure | Results |
| A7 | isopropanol | At the beginning, a | low |
| | | colloidal substance | degree of |
| | | was obtained, after | crystallinity |
| | | stirring at room | |
| | | temperature for 2 | |
| | | hours, it turned to a | |
| | | suspension, and it | |
| | | was kept as such | |
| | | after stirring for 18 h. | |
| B7 | butanone | At the beginning, a | oxalate |
| | | colloidal substance | crystalline |
| | | was obtained, after | form I |
| | | stirring at room | |
| | | temperature for 2 | |
| | | hours, it remained a | |
| | | colloidal substance. | |
| | | It was transferred | |
| | | to a 50° C. environment, | |
| | | and then adding 1 mL | |
| | | of n-heptane, it was | |
| | | stirred for 7 hours to | |
| | | obtain a suspension. | |
| C7 | 2- | After stirring for 20 h, | low |
| | methyl- | it remained a solution, | degree of |
| | tetrahydrofuran | adding 1 mL of n-heptane, | crystallinity |
| | | after stirring for 7 | |
| | | hours, which turned | |
| | | to a suspension. | |
| D7 | acetonitrile/ | At the beginning, a | approximate |
| | water, 19/1 | colloidal substance | amorphous |
| | | was obtained, after | |
| | | stirring at room | |
| | | temperature for 2 hours, | |
| | | it turned to a thick | |
| | | suspension. After adding | |
| | | 0.2 mL of solvent and | |
| | | stirring for 18 hours, | |
| | | finally, a suspension | |
| | | was obtained. | |

Oxalate crystalline form I (batch No.: A16498-0041B7): about 25 mg of starting material free base (batch No.: A16803-008S3) was weighed, 0.5 mL of butanone was added at room temperature (~24° C.) and the mixture was stirred for about 10 minutes to obtain a clear solution. 4.5 mg (1.1 equivalents) of oxalic acid solid was weighed and added to a free base solution, at the beginning, a colloidal substance was obtained, after stirring at room temperature for 20 hours, it remained a colloidal substance. It was transferred to a 50° C. environment, and then 1 mL of n-heptane was added, the mixture was stirred for 7 hours to obtain a suspension. The suspension was filtered by using a syringe equipped with 0.22 μm microporous filter membrane to obtain a solid sample, and the solid was dried under vacuum at 40° C. for 5 hours to obtain the sample. It was identified as oxalate crystalline form I by X-ray powder diffraction.

### 2.7 Maleate Crystalline Form I

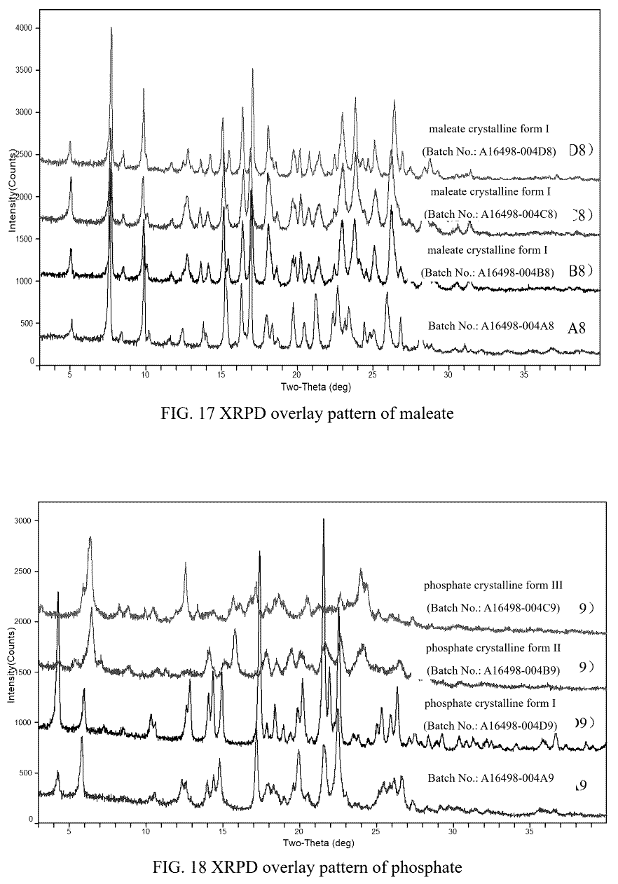

One maleate crystalline form was obtained by salt formation of free base and maleic acid, named maleate crystalline form I. Maleate crystalline form I can be obtained in butanone, 2-methyltetrahydrofuran or acetonitrile/water (19/1, v/v) system. (The results were shown in Table 11). The XRPD pattern was shown in FIG. 17.

TABLE 11
Preparation of maleate

| Batch | | | |
|---|---|---|---|
| No. | | | |
| A16498- | Solvent | | XPRD |
| 004 | (v/v) | Experimental procedure | Results |
| A8 | isopropanol | At the beginning, a suspension | similar to |
| | | was obtained, and after | maleate |
| | | stirring for 20 hours, it | crystalline |
| | | was kept as such. | form I |
| B8 | butanone | At the beginning, a suspension | maleate |
| | | was obtained, and after | crystalline |
| | | stirring for 20 hours, it | form I |
| | | was kept as such. | |
| C8 | 2- | At the beginning, a solution | maleate |
| | methyl- | was obtained, solid precipitates | crystalline |
| | tetrahydrofuran | were observed after stirring for | form I |
| | | 2 min, and after stirring for 20 | |
| | | hours, it remained a suspension. | |
| D8 | acetonitrile/ | At the beginning, a suspension | maleate |
| | water, | was obtained, and after | crystalline |
| | 19/1 | stirring for 20 hours, it | form I |
| | | was kept as such. | |

Maleate crystalline form I (batch No.: A16498-004B8): about 25 mg of starting material free base (batch No.: A16803-008S3) was weighed, 0.5 mL of butanone was added at room temperature (~24° C.) and the mixture was stirred for about 10 minutes to obtain a clear solution. 5.8 mg (1.1 equivalents) of maleic acid solid was weighed and added to a free base solution to obtain a suspension, after stirring for 20 hours, it was kept as such. The suspension was filtered by using a syringe equipped with 0.22 μm microporous filter membrane to obtain a solid sample, and the solid was dried under vacuum at 40° C. for 5 hours to obtain the sample. It was identified as maleate crystalline form I by X-ray powder diffraction.

Maleate crystalline form I (batch No.: A16498-004C8): a solution of free base (batch No.: A16803-008S3) in 2-methyltetrahydrofuran with a concentration of 60 mg/mL was prepared, and 416 μL of the solution was taken into a glass vial. 5.8 mg (1.1 equivalents) of maleic acid solid was added weighed and to a free base solution to obtain a solution, solid precipitates were observed after stirring for 2 min, and after stirring for 20 hours, it remained a suspension. The suspension was filtered by using a syringe equipped with 0.22 μm microporous filter membrane to obtain a solid sample, and the solid was dried under vacuum at 40° C. for 5 hours to obtain the sample. It was identified as maleate crystalline form I by X-ray powder diffraction.

Maleate crystalline form I (batch No.: A16498-004d8): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. 5.8 mg (1.1 equivalents) of maleic acid solid was weighed and added to a free base suspension to obtain a suspension, after stirring for 20 hours, it remained a suspension. The suspension was filtered by using a syringe equipped with 0.22 m microporous filter membrane to obtain a solid sample, and the solid was dried under vacuum at 40° C. for 5 hours to obtain the sample. It was identified as maleate crystalline form I by X-ray powder diffraction.

### 2.8 Phosphate

Three phosphate crystalline forms (crystalline form I, crystalline form II, and crystalline form III) were obtained by salt formation of free base as the raw material and 1.1 equivalents of phosphoric acid. (The results were shown in Table 12). The XRPD pattern was shown in FIG. 18.

TABLE 12
Preparation of phosphate

| Batch No. | | | |
|---|---|---|---|
| A16498- | Solvent | | XPRD |
| 004 | (v/v) | Experimental procedure | Results |
| A9 | isopropanol | At the beginning, the | similar to |
| | | solution was almost clear, | phosphate |
| | | and after stirring at room | crystalline |
| | | temperature for 2 hours, | form I |
| | | it was turned to a thick | |
| | | suspension. After adding | |
| | | 0.2 mL of isopropanol, | |
| | | after stirring for 18 hours, a | |
| | | suspension was obtained. | |
| B9 | butanone | After stirring at room | phosphate |
| | | temperature for 20 hours, | crystalline |
| | | the suspension was kept as | form II |
| | | such. | |
| C9 | 2-methyltetra- | At the beginning, a | phosphate |
| | hydrofuran | solution was obtained, | crystalline |
| | | and after stirring for 2 | form III |
| | | hours, it remained a | |
| | | solution. After stirring | |
| | | for 18 hours, a suspension | |
| | | was obtained. | |
| D9 | acetonitrile/ | After stirring for 2 hours, | phosphate |
| | water, 19/1 | a thick suspension was | crystalline |
| | | obtained, and adding | form I |
| | | 0.2 mL of solvent, a | |
| | | suspension was | |
| | | obtained after stirring | |
| | | for 18 hours | |

### 2.8.1 Phosphate Crystalline Form I

Phosphate crystalline form I (batch No.: A16498-004D9): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. 58 mg of phosphoric acid was added to 0.5 mL of acetonitrile/water (19/1, v/v), mixing well, and then 56 μL of the mixture (containing 1.1 equivalents of phosphoric acid) was added to the free base suspension. At the beginning, there was flocculent solid precipitation, after stirring for 2 hours, a thick suspension was obtained, and 0.2 mL of solvent was added, a suspension was obtained after stirring for 18 hours. The suspension was filtered by using a syringe equipped with 0.22 μm microporous filter membrane to obtain a solid sample, and the solid was dried under vacuum at 40° C. for 5 hours to obtain the sample. It was identified as phosphate crystalline form I by X-ray powder diffraction.

### 2.8.2 Phosphate Crystalline Form II

Phosphate crystalline form II (batch No.: A16498-004B9): about 25 mg of starting material free base (batch No.: A16803-008S3) was weighed, 0.5 mL of butanone was added at room temperature (~24° C.) and the mixture was stirred for about 10 minutes to obtain a clear solution. 58 mg of phosphoric acid was added to 0.5 mL of butanone, mixing well, and then 56 μL of the mixture (containing 1.1 equivalents of phosphoric acid) was added to the free base suspension. After stirring at room temperature for 20 hours, the suspension was kept as such. The suspension was filtered by using a syringe equipped with 0.22 μm microporous filter membrane to obtain a solid sample, and the solid was dried under vacuum at 40° C. for 5 hours to obtain the sample. It was identified as phosphate crystalline form II by X-ray powder diffraction.

### 2.8.3 Phosphate Crystalline Form III

Phosphate crystalline form III (batch No.: A16498-004C9): a solution of free base (batch No.: A16803-008S3) in 2-methyltetrahydrofuran with a concentration of 60 mg/mL was prepared, and 416 μL of the solution was taken into a glass vial. 58 mg of phosphoric acid was added to 0.5 mL of 2-methyltetrahydrofuran, after mixing well, 56 μL of the mixture (containing 1.1 equivalents of phosphoric acid) was added to a free base solution to obtain a solution. After stirring for 2 hours, it remained a solution. After stirring for 18 hours, a suspension was obtained. The suspension was filtered by using a syringe equipped with 0.22 μm microporous filter membrane to obtain a solid sample, and the solid was dried under vacuum at 40° C. for 5 hours to obtain the sample. It was identified as phosphate crystalline form III by X-ray powder diffraction.

### 2.8.4 Scale-Up Production of Phosphate Crystalline Form I

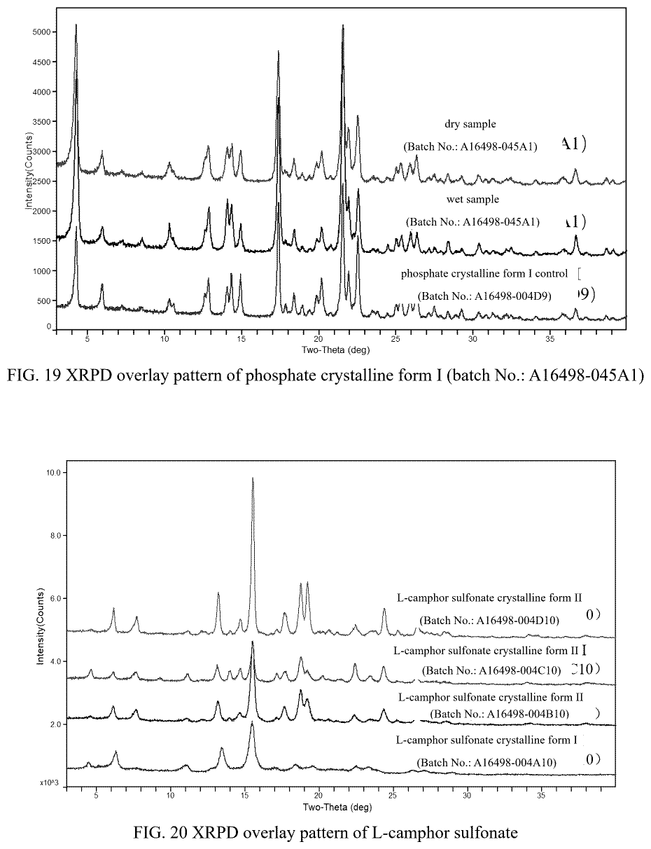

About 350 mg of free base (batch No.: A16803-008S3) was weighed, and 7 mL of acetonitrile/water (19/1, v/v) was added, the mixture was stirred at room temperature (~21° C.) to obtain a suspension, and 1.1 equivalents of phosphoric acid solution (81 mg of phosphoric acid was diluted with 2 mL of acetonitrile/water, 19/1, v/v). At the beginning, the suspension became almost clear. After stirring for 10 minutes, a thick suspension was obtained and 6 mL of acetonitrile/water (19/1, v/v) was added. Finally, after stirring at room temperature for 16 hours, a suspension was obtained. The solid was collected by suction filtration with a vacuum pump and dried under vacuum at 40° C. for 4 hours. A sample (batch No.: A16498-045A1) was obtained in a yield of 93%. It was identified as phosphate crystalline form I by X-ray powder diffraction (FIG. 19).

### 2.9 L-Camphor Sulfonate

Two crystalline forms of L-camphor sulfonate were obtained by salt formation of free base and L-camphor sulfonic acid, named L-camphor sulfonate crystalline form I and L-camphor sulfonate crystalline form II respectively. (The results were shown in Table 13). The XRPD pattern was shown in FIG. 20.

TABLE 13
Preparation of L-camphor sulfonate

| Batch | | | |
|---|---|---|---|
| No. | | | |
| A16498- | Solvent | | |
| 004 | (v/v) | Experimental procedure | XPRD Results |
| A10 | isopropanol | At the beginning, a colloidal | L-camphor |
| | | substance was obtained, | sulfonate |
| | | after stirring at ordinary | crystalline |
| | | temperature for 20 hours, | form I |
| | | it was kept as such. | |
| | | Adding 0.4 mL of | |
| | | isopropanol, and it was | |
| | | stirred at 50° C. for 7 hours | |
| | | to obtain a suspension | |
| B10 | butanone | At the beginning, a colloidal | L-camphor |
| | | substance was obtained, | sulfonate |
| | | after stirring at room | crystalline |
| | | temperature for 20 hours, | form |
| | | it was kept as such. Adding | II |
| | | 0.4 mL of butanone, | |
| | | and it was stirred at | |
| | | 50° C. for 7 hours | |
| | | to obtain a suspension | |
| C10 | 2- | At the beginning, a solution | L-camphor |
| | methyltetra- | was obtained, and after | sulfonate |
| | hydrofuran | stirring for 2 hours, | crystalline |
| | | it turned to colloidal | form |
| | | substance. After stirring at | II |
| | | room temperature for 18 | |
| | | hours, it was kept as such. | |
| | | Adding 0.6 mL of | |
| | | 2-methyltetrahydrofuran | |
| | | and the mixture was | |
| | | stirred at 50° C., a | |
| | | suspension was obtained | |
| | | after stirring for 7 hours. | |
| D10 | acetonitrile/ | At the beginning, a colloidal | L-camphor |
| | water, | substance was obtained, | sulfonate |
| | 19/1 | after stirring at room | crystalline |
| | | temperature for 20 hours, | form |
| | | it was kept as such. Adding | II |
| | | 0.6 mL of solvent and | |
| | | it was stirred at 50° C., | |
| | | after stirring for 7 hours, | |
| | | a suspension was obtained. | |

### 2.9.1 L-Camphor Sulfonate Crystalline Form I

L-camphor sulfonate crystalline form I (batch No.: A16498-004A10): about 25 mg of starting material free base (batch No.: A16803-008S3) was weighed, 0.5 mL of isopropanol was added at room temperature (~24° C.) and the mixture was stirred for about 10 minutes to obtain a white suspension. 11.6 mg (1.1 equivalents) of L-camphor sulfonic acid solid was weighed and added to a suspension of the active pharmaceutical ingredient. At the beginning, a colloidal substance was obtained, after stirring at room temperature for 20 hours, it was kept as such. 0.4 mL of isopropanol was added, after stirring for 7 hours at 50° C., a suspension was obtained. The suspension was filtered by using a syringe equipped with 0.22 μm microporous filter membrane to obtain a solid sample, and the solid was dried under vacuum at 40° C. for 5 hours to obtain the sample. It was identified as L-camphor sulfonate crystalline form I by X-ray powder diffraction.

### 2.9.2 L-Camphor Sulfonate Crystalline Form II

L-camphor sulfonate crystalline form II can be obtained in butanone, 2-methyltetrahydrofuran or acetonitrile/water (19/1, v/v) system.

L-camphor sulfonate crystalline form II (batch No.: A16498-004B10): about 25 mg of starting material free base (batch No.: A16803-008S3) was weighed, 0.5 mL of butanone was added at room temperature (~24° C.) and the mixture was stirred for about 10 minutes to obtain a clear solution. 11.6 mg (1.1 equivalents) of L-camphor sulfonic acid solid was weighed and added to the drug solution to obtain a colloidal substance, after stirring at room temperature for 20 hours, it remained a colloidal substance. 0.4 mL of butanone was added, after stirring for 7 hours at 50° C., a suspension was obtained. The suspension was filtered by using a syringe equipped with 0.22 μm microporous filter membrane to obtain a solid sample, and the solid was dried under vacuum at 40° C. for 5 hours to obtain the sample. It was identified as L-camphor sulfonate crystalline form II by X-ray powder diffraction.

L-camphor sulfonate crystalline form II (batch No.: A16498-004C10): a solution of free base (batch No.: A16803-008S3) in 2-methyltetrahydrofuran with a concentration of 60 mg/mL was prepared, and 416 μL of the solution was taken into a glass vial. 11.6 mg (1.1 equivalents) of L-camphor sulfonic acid solid was weighed and added to the drug solution. At the beginning, a solution was obtained, and after stirring for 2 hours, it turned to a colloidal substance. After stirring at room temperature for 18 hours, it was kept as such. 0.6 mL of 2-methyltetrahydrofuran was added, a suspension was obtained after stirring for 7 hours at 50° C. The suspension was filtered by using a syringe equipped with 0.22 μm microporous filter membrane to obtain a solid sample, and the solid was dried under vacuum at 40° C. for 5 hours to obtain the sample. It was identified as L-camphor sulfonate crystalline form II by X-ray powder diffraction.

L-camphor sulfonate crystalline II (batch No.: A16498-004D10): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. 11.6 mg (1.1 equivalents) of L-camphor sulfonic acid was added to the suspension. At the beginning, a colloidal substance was obtained, after stirring at room temperature for 20 hours, it was kept as such. 0.6 mL of acetonitrile/water (19/1, v/v) was added, after stirring for 7 hours at 50° C., a suspension was obtained. The suspension was filtered by using a syringe equipped with 0.22 μm microporous filter membrane to obtain a solid sample, and the solid was dried under vacuum at 40° C. for 5 hours to obtain the sample. It was identified as L-camphor sulfonate crystalline form II by X-ray powder diffraction.

### 2.10 Gentisate

It was attempted to form salt of free base as a raw material with gentisic acid, but gentisate was not obtained. The detailed information and results were summarized in Table 14 and FIG. 21.

TABLE 14
Preparation of gentisate

| Batch | | | |
|---|---|---|---|
| No. | | | |
| A16498- | Solvent | | XPRD |
| 004 | (v/v) | Experimental procedure | Results |
| A11 | isopropanol | After stirring for 20 hours, | free base |
| | | the suspension | crystalline |
| | | was kept as such. | form II |
| B11 | butanone | At the beginning, a solution | N/A |
| | | was obtained, and after | |
| | | stirring for 20 hours, it was | |
| | | kept as such. Adding | |
| | | 1 mL of toluene and | |
| | | stirring for 2 days, and | |
| | | no solid precipitated. | |
| C11 | 2- | After stirring for 20 hours, | free base |
| | methyl- | the solution was nearly clear, | crystalline |
| | tetrahydrofuran | 1 mL of n-heptane was added, | form I |
| | | and after stirring for 7 hours, | |
| | | a solid precipitated. | |
| D11 | acetonitrile/ | After stirring for 20 | free base |
| | water, 19/1 | hours, the suspension | crystalline |
| | | was kept as such. | form IV |

### 2.11 Tartrate Crystalline Form I

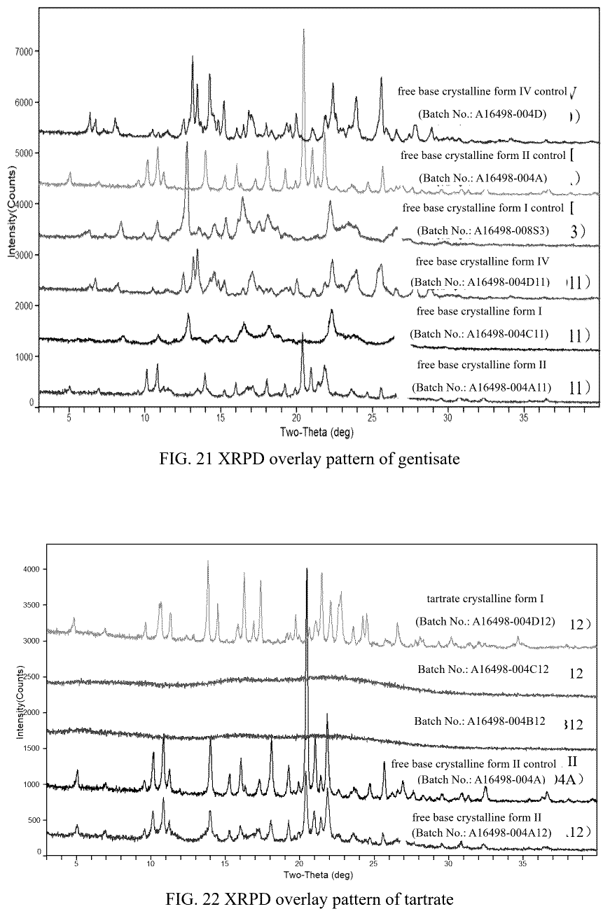

One tartrate crystalline form was obtained by salt formation of free base and tartaric acid, named tartrate crystalline form I. (The results were shown in Table 15). The XRPD pattern was shown in FIG. 22.

TABLE 15
Preparation of tartrate

| Batch | | | |
|---|---|---|---|
| No. | | | |
| A16498- | Solvent | | XPRD |
| 004 | (v/v) | Experimental procedure | Results |
| A12 | iso- | At the beginning, a thick suspension | free base |
| | propanol | was obtained and 0.2 mL of isopropanol | crystalline |
| | | was added. After stirring for 2 hours, a | form II |
| | | suspension was obtained, and after | |
| | | stirring for 18 hours, it was kept as such. | |
| B12 | butanone | After stirring for 20 hours, the solution | amorphous |
| | | was nearly clear, 1 mL of n-heptane was | |
| | | added and a solid precipitated, and the | |
| | | mixture remained a suspension after | |
| | | stirring for 7 hours. | |
| C12 | 2- | After stirring for 20 hours, the solution | amorphous |
| | methyl- | was nearly clear, 1mL of n-heptane was | |
| | tetra- | added and a solid and oil precipitated, | |
| | hydro- | and the mixture turned to a suspension | |
| | furan | after stirring for 7 hours. | |
| D12 | aceto- | At the beginning, a suspension was | tartrate |
| | nitrile/ | obtained, after stirring for 2 hours, it | crystalline |
| | water, | turned to a thick suspension, and adding | form I |
| | 19/1 | 0.2 mL of ACN/ water, finally a | |
| | | suspension was obtained after stirring | |
| | | for 18 hours. | |

Tartrate crystalline form I (batch No.: A16498-004D12): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. 7.5 mg (1.1 equivalents) of tartaric acid solid was weighed and added to the suspension. At the beginning, a suspension was obtained, after stirring for 2 hours, it turned to a thick suspension, and 0.2 mL of acetonitrile/water (19/1, v/v) was added, finally a suspension was obtained after stirring for 18 hours. The suspension was filtered by using a syringe equipped with 0.22 μm microporous filter membrane to obtain a solid sample, and the solid was dried under vacuum at 40° C. for 5 hours to obtain the sample. It was identified as tartrate crystalline form I by X-ray powder diffraction.

### 2.12 Fumarate Crystalline Form I

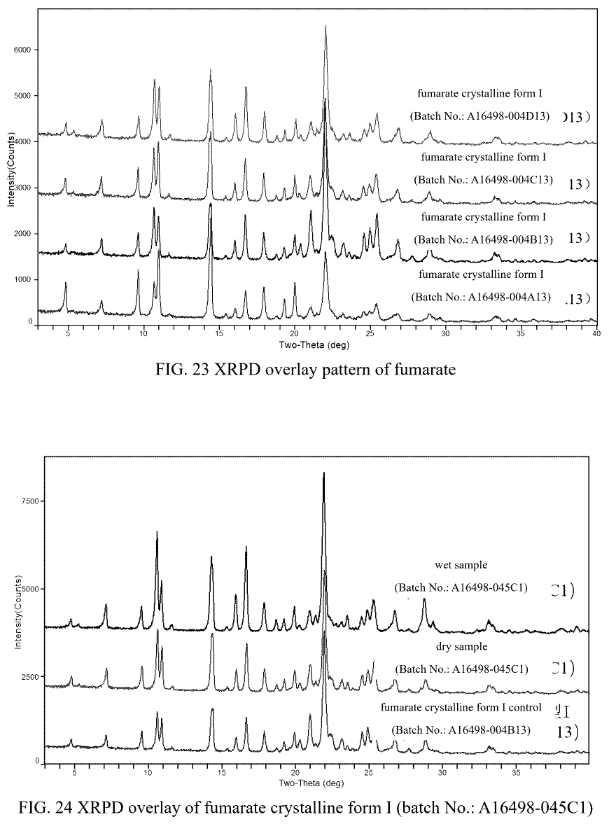

A fumarate crystalline form was obtained by salt formation of free base and 1.1 equivalents of fumaric acid, named fumarate crystalline form I. All the fumarate obtained in four solvent systems of isopropanol, butanone, 2-methyltetrahydrofuran and acetonitrile/water (19/1, v/v) are fumarate crystal form I, which means that only one compound A fumarate crystalline form was formed under multiple crystallization systems. (The results were shown in Table 16). The XRPD pattern was shown in FIG. 23.

TABLE 16
Preparation of fumarate

| Batch | | | |
|---|---|---|---|
| No. | | | |
| A16498- | Solvent | | XPRD |
| 004 | (v/v) | Experimental procedure | Results |
| A13 | iso- | At the beginning, a suspension was | fumarate |
| | propanol | obtained, after stirring for | crystalline |
| | | 2 hours, it turned to a thick | form I |
| | | suspension, and adding 0.2 mL of | |
| | | isopropanol, finally a suspension was | |
| | | obtained after stirring for 18 hours. | |
| B13 | butanone | At the beginning, a solution | fumarate |
| | | was obtained, solid precipitates | crystalline |
| | | were observed after 2 min. | form I |
| | | After stirring for 2 hours, | |
| | | the suspension became thick, | |
| | | adding 0.2 mL of butanone. | |
| | | After stirring for 18 hours, | |
| | | a suspension was finally obtained. | |
| C13 | 2- | At the beginning, a solution | fumarate |
| | methyl | was obtained, and then solids | crystalline |
| | tetra- | precipitated. After stirring | form I |
| | hydro- | for 2 hours, the suspension | |
| | furan | became thick, adding 0.2 mL | |
| | | of 2-methyltetrahydrofuran. | |
| | | After stirring for 18 hours, | |
| | | a suspension was finally obtained. | |
| D13 | aceto- | At the beginning, a thick | fumarate |
| | nitrile/ | suspension was obtained and | crystalline |
| | water, | 0.6 mL of ACN/water was | form I |
| | 19/1 | added. After stirring for 20 hours, a | |
| | | suspension was finally obtained. | |

Fumarate crystalline form I (batch No.: A16498-004A13): about 25 mg of starting material free base (batch No.: A16803-008S3) was weighed, 0.5 mL of isopropanol was added at room temperature (~24° C.) and the mixture was stirred for about 10 minutes to obtain a white suspension. 5.8 mg (1.1 equivalents) of fumaric acid solid was weighed and added to a suspension of the active pharmaceutical ingredients. At the beginning, a suspension was obtained, and after stirring for 2 hours, the suspension became thick. 0.2 mL of isopropanol was added, after stirring at room temperature for 18 hours, finally, a suspension was obtained. The suspension was filtered by using a syringe equipped with 0.22 μm microporous filter membrane to obtain a solid sample, and the solid was dried under vacuum at 40° C. for 5 hours to obtain the sample. It was identified as fumarate crystalline form I by X-ray powder diffraction.

Fumarate crystalline form I (batch No.: A16498-0041B13): about 25 mg of starting material free base (batch No.: A16803-008S3) was weighed, 0.5 mL of butanone was added at room temperature (~24° C.) and the mixture was stirred for about 10 minutes to obtain a clear solution. 5.8 mg (1.1 equivalents) of fumaric acid solid was weighed and added to a solution of the active pharmaceutical ingredients. At the beginning, a solution was obtained, solid precipitates were observed after 2 min. After stirring for 2 hours, the suspension became thick, 0.2 mL of butanone was added. After stirring for 18 hours, finally, a suspension was obtained. The suspension was filtered by using a syringe equipped with 0.22 μm microporous filter membrane to obtain a solid sample, and the solid was dried under vacuum at 40° C. for 5 hours to obtain the sample. It was identified as fumarate crystalline form I by X-ray powder diffraction.

Fumarate crystalline form I (batch No.: A16498-004C13): a solution of free base (batch No.: A16803-008S3) in 2-methyltetrahydrofuran with a concentration of 60 mg/mL was prepared, and 416 μL of the solution was taken into a glass vial. 5.8 mg (1.1 equivalents) of fumaric acid solid was weighed and added to a solution of the active pharmaceutical ingredients. At the beginning, a solution was obtained, and then solids precipitated. After stirring for 2 hours, the suspension became thick, 0.2 mL of 2-methyltetrahydrofuran was. After stirring for 18 hours, a suspension was finally obtained. The suspension was filtered by using a syringe equipped with 0.22 μm microporous filter membrane to obtain a solid sample, and the solid was dried under vacuum at 40° C. for 5 hours to obtain the sample. It was identified as fumarate crystalline form I by X-ray powder diffraction.

Fumarate crystalline form I (batch No.: A16498-004D13): about 25 mg of starting material free base (batch No.: A16803-008S3) was weighed, 0.5 mL of acetonitrile/water (19/1, v/v) was added at room temperature (~24° C.) and the mixture was stirred for about 10 minutes to obtain a white suspension. After adding 5.8 mg (1.1 equivalents) of fumaric acid solid, the suspension became thick. 0.6 mL of acetonitrile/water (19/1, v/v) was added, finally a suspension was obtained after stirring for 20 hours. The suspension was filtered by using a syringe equipped with 0.22 μm microporous filter membrane to obtain a solid sample, and the solid was dried under vacuum at 40° C. for 5 hours to obtain the sample. It was identified as fumarate crystalline form I by X-ray powder diffraction.

### Scale-Up Production of Fumarate Crystalline Form I

Approximately 350 mg of the free base (batch No.: A16803-008S3) was weighed out and dissolved in 7 mL of butanone at room temperature (~21° C.) and 1.1 equivalents of fumaric acid solid (82 mg) was added. At the beginning, after stirring for 2 minutes a solution was obtained. Fumarate crystalline form I seed crystals were then added and stirred for about 3 minutes to give a thick suspension. Then 2 mL of butanone was added and after stirring at room temperature for 16 hours a suspension was obtained. The solid was collected by suction filtration with a vacuum pump and dried under vacuum at 40° C. for 4 hours. A sample (batch No.: A16498-045C1) was obtained with a yield of 85% and identified as fumarate crystalline form I by X-ray powder diffraction (FIG. 24).

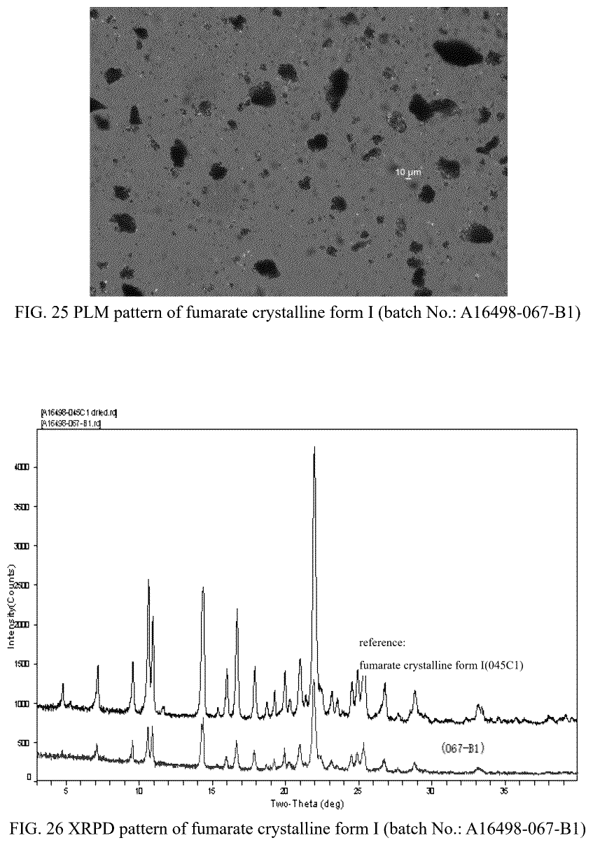

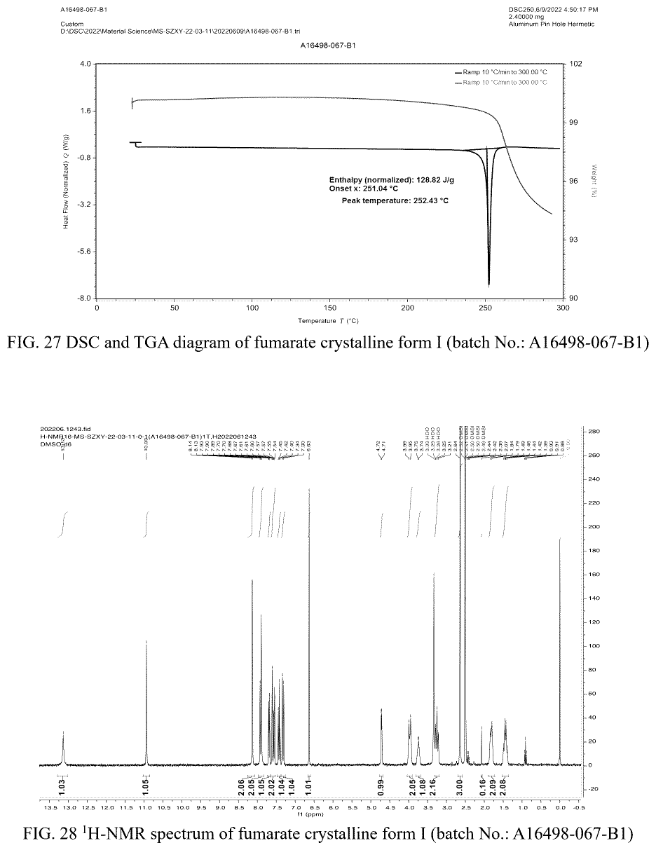

At room temperature, 3 g of free base form I was added to MEK (80 mL) to a clear solution, then fumaric acid (1.1 eq.) and seed crystals (fumarate form I, batch No.: A16498-045C1) were added to the solution and stirred at room temperature for 20 hours. The solid was collected by filtration, and an excessive amount of fumaric acid was found to remain. The solid was added to MEK (40 mL) and stirred at room temperature for 4 h. The suspension was filtered and the filter cake was dried at 40° C. for 3 hours. Fumarate crystalline form I (batch No. A16498-067-B1) was successfully prepared in a yield of 81.5% (2.7 g). The characterization data are shown in FIGS. 25-28.

### 3. Related Properties of Pharmaceutically Acceptable Salts of Compound A

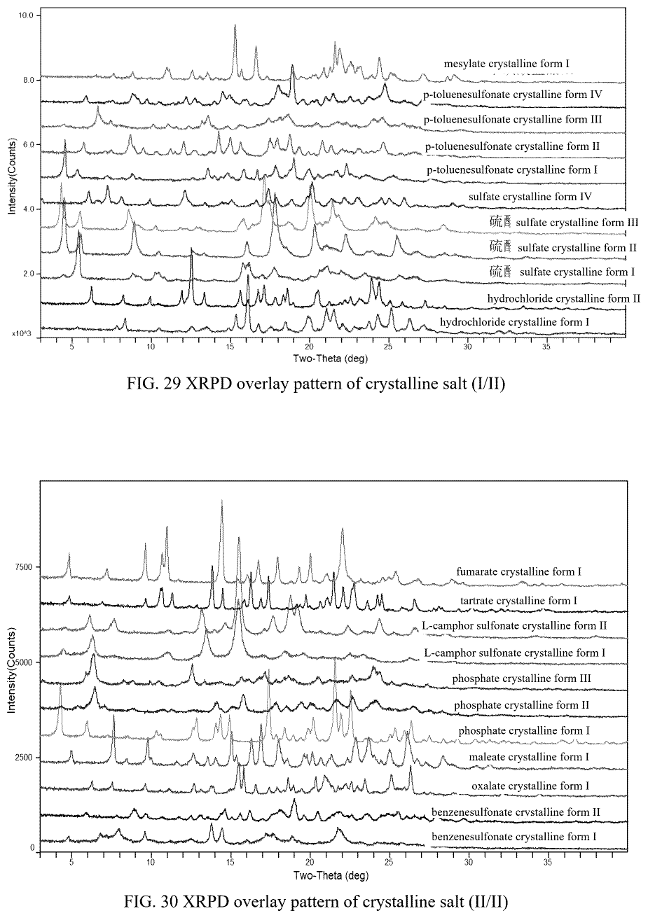

Compound A defined as free base crystalline form I is an anhydrous form with a melting point of 245° C. The salt form screening was carried out to obtain eleven crystalline salts and twenty-two crystalline forms thereof, including hydrochloride crystalline form II, sulfate crystalline form I/II/III/IV, p-toluenesulfonate crystalline form I/II/III/IV, mesylate crystalline form I, besylate crystalline form I/II, oxalate crystalline form I, maleate crystalline form I, phosphate crystalline form IIII L-camphorsulfonate crystalline form II, tartrate crystalline form I and fumarate crystalline form I. Characterization data of the anhydrous crystalline form and the possible anhydrous crystalline form sample were shown in table 16, FIG. 29 and FIG. 30.

TABLE 16
Summary of solid-state characterization data for free base and crystalline salts

| | | | DSC | DVS | |
|---|---|---|---|---|---|
| | | | Melting | The weight | |
| | | | point/Peak | gains by | |
| | | | (endothermic | vapor | |
| Solid form | | TGA | Peak° C.), | sorption % | |
| base:acid^{a} | Crystallinity | Weight loss %/ | enthalpy | @80, 90 | |
| solvate | (XPRD) | temperature (° C.) | (J/g) | RH % | Notes |
| Free base | Moderate | 0.2/170-250 | 245/247, 64 | 0.8, 0.9 | starting |
| crystalline form I | | | | slight | material |
| Anhydrous | | | | hygroscopic | 0.2 wt. % |
| crystalline form | | | | | MTBE |
| Free base | High | 8.5/100-150 | Multiple | / | ~0.8 eq. |
| crystalline form II | | | endothermic | | IPA |
| IPA solvate | | | peaks | | |
| Free base | Moderate | 0.3/100-225 | 234/238, 8 | / | 0.2 wt. % 2- |
| crystalline form III | | | 245/247, 61 | | Me THF |
| Anhydrous | | | | | |
| crystalline form | | | | | |
| Free base | Moderately | 1.9/80-150 | 89/120, 17 | / | ~0.3 eq. |
| crystalline form IV | higher | | 161/167, 18 | | ACN |
| ACN solvate | | | 246/247, 64 | | |
| Hydrochloride | Moderately | 1.9/30-150 | 55/90, 26 | / | 0.1 wt. % |
| crystalline form I | higher | 1.0/150-205 | 171/184, 56 | | ACN |
| 1:1 | | | (m/d) ^{b} | | |
| hydrate | | | | | |
| Hydrochloride | High | 7.0/60-120 | 94/104, 46 | / | ~0.6 eq. |
| crystalline form II | | 2.3/175-245 | 209/224, 102 | | MEK |
| MEK solvate | | | (m/d) ^{b} | | |
| Sulfate crystalline | Moderate | 1.6/37-90 | Multiple | / | 2.1 wt. % |
| form I | | 4.0/90-175 | endothermic | | IPA and 0.2 |
| IPA solvate/hydrate | | 2.0/175-220 | peaks | | wt. % n- |
| | | | | | heptane |
| Sulfate crystalline | Moderate | 0.8/40-100 | 30/62, 10 | / | 0.5 eq. |
| form II | | 6.2/100-180 | 144/146, 75 | | MEK |
| MEK | | | | | |
| solvate/hydrate | | | | | |
| Sulfate crystalline | Moderate | 1.3/50-120 | Multiple | / | ~0.5 eq. 2- |
| form III | | 7.7/120-200 | endothermic | | Me THF |
| 2-Me THF | | | peaks | | |
| solvate/hydrate | | | | | |
| Sulfate crystalline | Moderate | 2.8/25-120 | Multiple | / | 0.4 wt. % |
| form IV | | 2.1/120-220 | endothermic | | ACN |
| hydrate | | | peaks | | |
| p-Toluenesulfonate | Moderately | 0.8/27-75 | 26/45, 15 | / | ~0.2 eq. |
| crystalline form I | higher | 1.2/90-160 | 92/120, 3 | | IPA |
| 1:1 | | | 157/165, 32 | | |
| IPA solvate/hydrate | | | | | |
| p-Toluenesulfonate | Moderately | 1.8/40-150 | 46/104, 10 | / | ~0.2 eq. |
| crystalline form II | higher | 0.8/150-200 | 169/183, 43 | | MEK |
| 1:0.9 | | | (m/d) ^{b} | | |
| MEK solvate | | | | | |
| p-Toluenesulfonate | Moderate | 0.7/29-85 | 27/39, 12 | / | ~0.3 eq. 2- |
| crystalline form III | | 3.1/85-150 | 89/114, 12 | | Me THF |
| 1:1 | | | 188/193, 33 | | |
| 2-Me THF | | | | | |
| solvate/hydrate | | | | | |
| p-Toluenesulfonate | Moderate | 1/35-120 | 42/73, 11 | / | 0.2 wt. % |
| crystalline form IV | | | 174/184, 53 | | ACN |
| 1:0.9 | | | | | |
| hydrate | | | | | |
| Mesylate | High | 0.4/24-65 | 239/241, 56 | / | the |
| crystalline form I | | 0.8/100-200 | (m/d) ^{b} | | crystalline |
| 1:1 | | | | | form is not |
| Anhydrous | | | | | stable under |
| crystalline form | | | | | high |
| | | | | | humidity |
| | | | | | conditions |
| | | | | | 0.8 wt. % |
| | | | | | IPA |
| Besylate crystalline | Moderate | 0.5/28-90 | 41/62, 5 | / | / |
| form I | | | 180/186, 30 | | |
| 1:0.9 | | | | | |
| hydrate | | | | | |
| Besylate crystalline | Moderate | 1.1/40-155 | 44/94, 8 | / | 0.9 wt. % |
| form II | | | 181/187, 51 | | ACN |
| 1:0.9 | | | | | |
| ACN | | | | | |
| solvate/anhydrous | | | | | |
| crystalline form | | | | | |
| Oxalate crystalline | Moderately | 0.4/25-80 | 27/36, 30 | / | 0.7 wt. % |
| form I | higher | | 178/183, 58 | | MEK |
| 1:1.3 | | | | | |
| Anhydrous | | | | | |
| crystalline form | | | | | |
| Maleate crystalline | High | 1.0/25-90 | 26/54, 36 | / | 2.1 wt. % |
| form I | | 2.1/120-190 | 193/195, 116 | | MEK |
| 1:1 | | | | | |
| hydrate/MEK | | | | | |
| solvate | | | | | |
| Phosphate | High | no apparent | 212/219, 135 | 1.3, 1.6 | / |
| crystalline form I | | weight loss | 255/267, 29 | slight | |
| 1:1 | | below 160° C. | | hygroscopic | |
| Anhydrous | | | | | |
| crystalline form | | | | | |
| Phosphate | Moderate | 2.4/30-100 | Multiple | / | 0.3 eq. |
| crystalline form II | | 2.1/100-150 | endothermic | | MEK |
| MEK | | | peaks | | |
| solvate/hydrate | | | | | |
| Phosphate | Moderate | 3.9/80-125 | 100/107, 19 | / | ~0.4 eq. 2- |
| crystalline form III | | 2/170-250 | 204/222, 93 | | Me THF |
| 2-Me THF solvate | | | | | |
| L-camphor | Moderate | 0.3/30-100 | 28/43, 10 | / | / |
| sulfonate | | | 226/235, 75 | | |
| crystalline form II | | | | | |
| 1:1 | | | | | |
| hydrate | | | | | |
| L-camphor | Moderately | 0.2/30-200 | 226/233, 67 | 0.8, 1.2 | / |
| sulfonate | higher | | (m/d) ^{b} | slight | |
| crystalline form II | | | | hygroscopic | |
| 1:1 | | | | | |
| Anhydrous | | | | | |
| crystalline form | | | | | |
| Tartrate crystalline | High | 2.8/30-110 | Multiple | / | / |
| form I | | | endothermic | | |
| 1:0.5 | | | peaks | | |
| hydrate | | | | | |
| Fumarate | High | 1.6/160-250 | 251/252, 129 | 0.2, 0.2 | / |
| crystalline form I | | | (m/d) ^{b} | almost non | |
| 1:0.5 | | | | hygroscopic | |
| Anhydrous | | | | | |
| crystalline form | | | | | |

^{a}Salt formation ratio; a salt formation ratio of inorganic salt was determined by ion chromatography, and salt formation ratio of organic salt was analyzed by ¹H-NMR
^{b)} m/d: Melting accompanied with decomposition

### 3.1 Short-Term Physical Stability Analysis of Anhydrous Crystal Forms

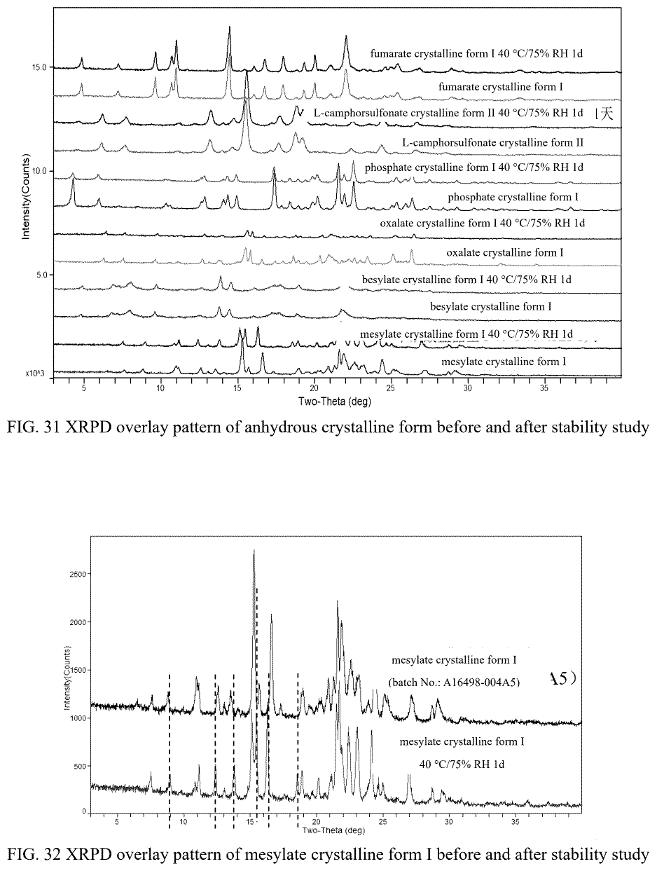

Mesylate crystalline form I, besylate crystalline form I, oxalate crystalline form I, phosphate crystalline form I, L-camphorsulfonate crystalline form II, and fumarate crystalline form I were placed in a high humidity environment (40° C./75% RH, open) for 1 day. The short-term physical stability results of these anhydrous crystalline form samples were shown in Table 17 and FIG. 31. In addition to the mesylate crystalline form I, the other anhydrous crystalline forms were stable under high humidity conditions. As shown in FIG. 32, a new crystalline form of mesylate was found, indicating that mesylate crystalline form I was unstable and crystal transformation occurred while standing for one day under high humidity conditions.

TABLE 17
Evaluation results of short-term physical stability

| Solid form | Batch No. | XPRD Results |
|---|---|---|
| Mesylate crystalline form I | A16498-004A5 | new crystalline form |
| Besylate crystalline form I | A16498-004A6 | unchanged |
| Oxalate crystalline form I | A16498-004B7 | unchanged |
| Phosphate crystalline form I | A16498-004D9 | unchanged |
| L-camphor sulfonate | A16498-004B10 | unchanged |
| crystalline form II | | |
| Fumarate crystalline form I | A16498-004A13 | unchanged |

Because of appropriate solid-state properties of phosphate crystalline form I, L-camphorsulfonate crystalline form II and fumarate crystalline form I, their preparation scale was scaled up to 350 mg, respectively, for hygroscopicity evaluation. DVS results indicated that phosphate crystalline form I and L-camphorsulfonate crystalline form II are slightly hygroscopic and fumarate form crystalline I is almost non-hygroscopic. However, colloid formation is observed during the preparation of L-camphorsulfonate crystalline form II, presenting a potential risk during the manufacturing process.

Conclusion: It can be seen from the above test results that phosphate crystalline form I and fumarate crystalline form I have better solid-state properties, and have the beneficial properties of compatibility in the aspects of rationality of acid-base salt formation ratio, crystallinity, no solvation and hygroscopicity, and the fumaric acid crystalline form I also has the beneficial property of single crystalline form under various crystallization systems.

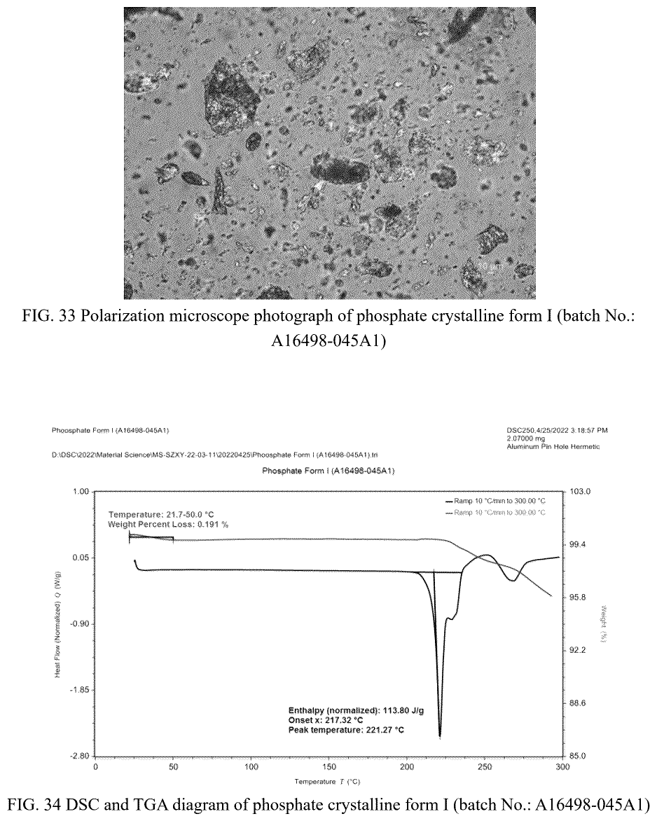

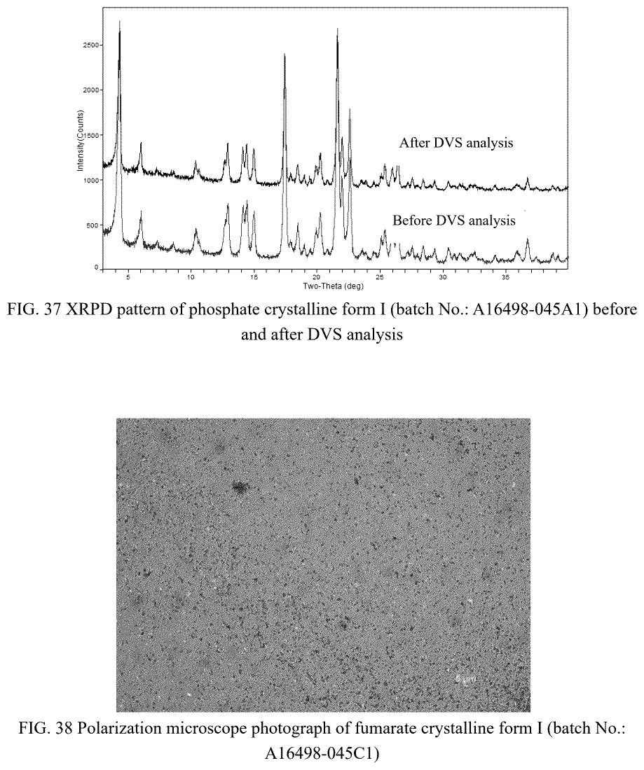

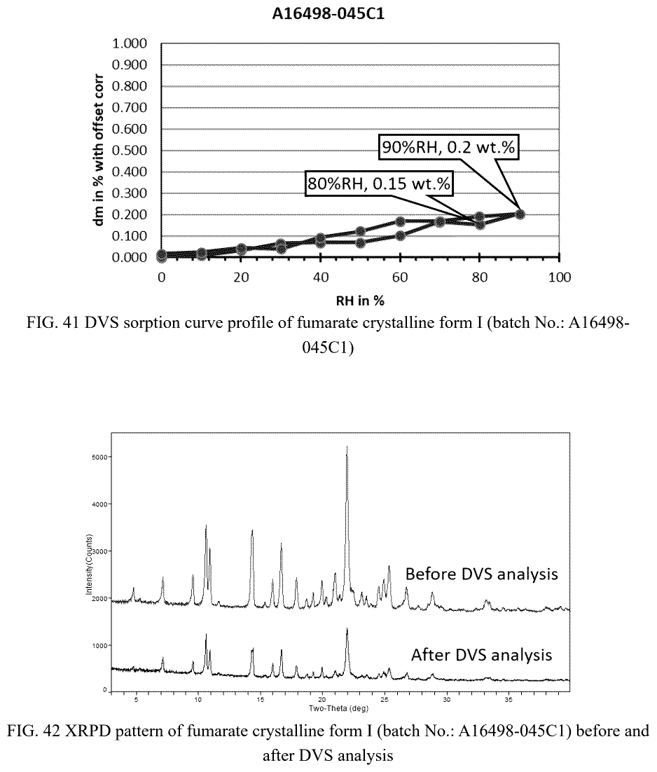

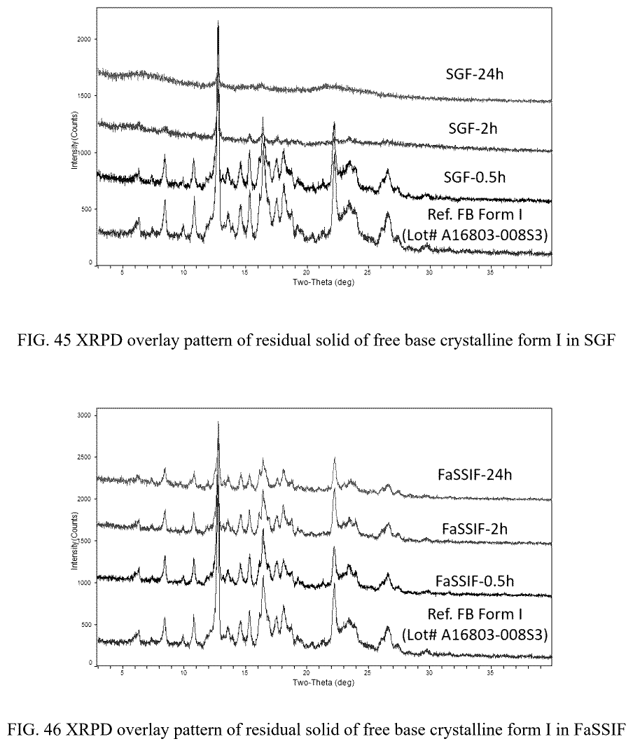

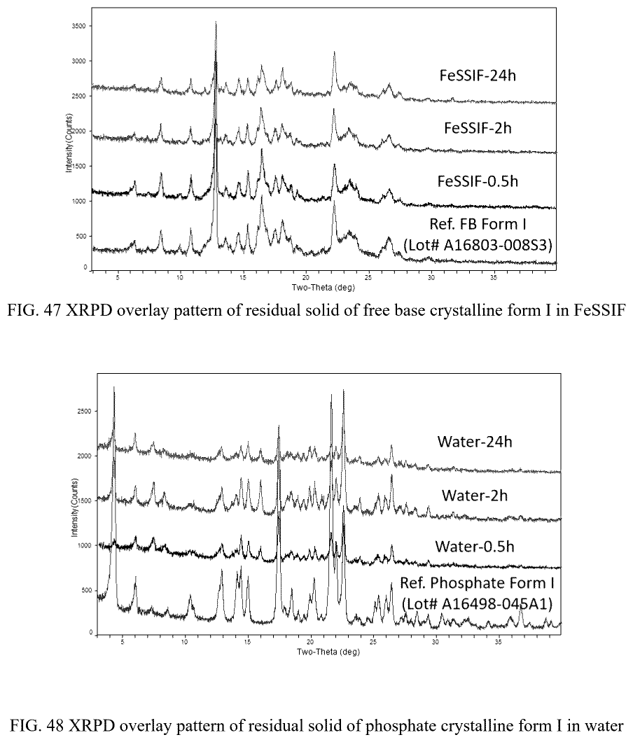

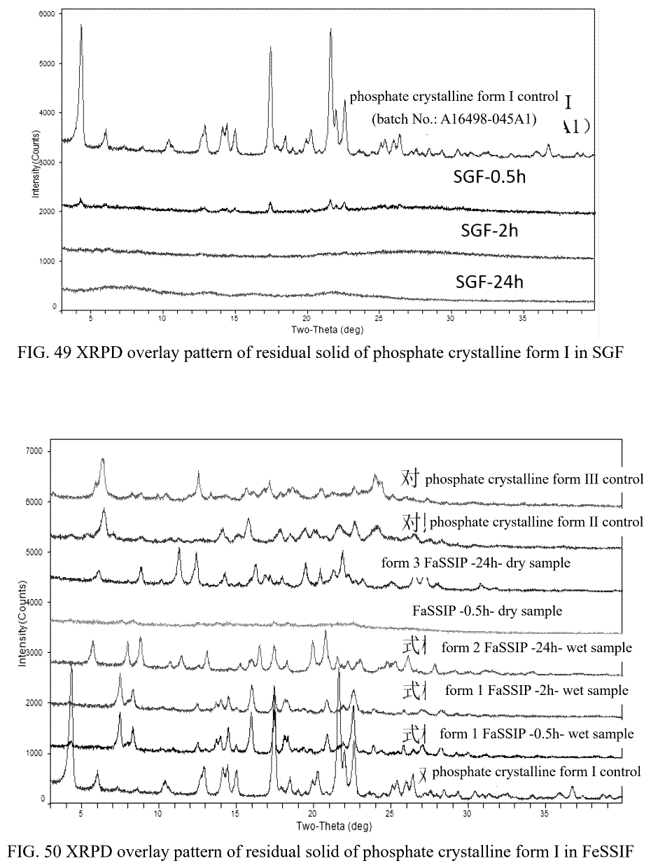

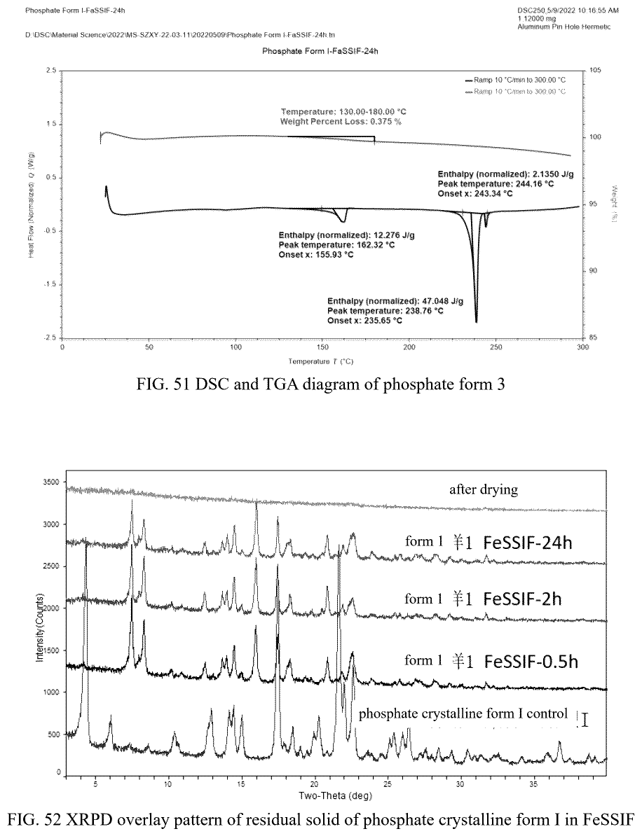

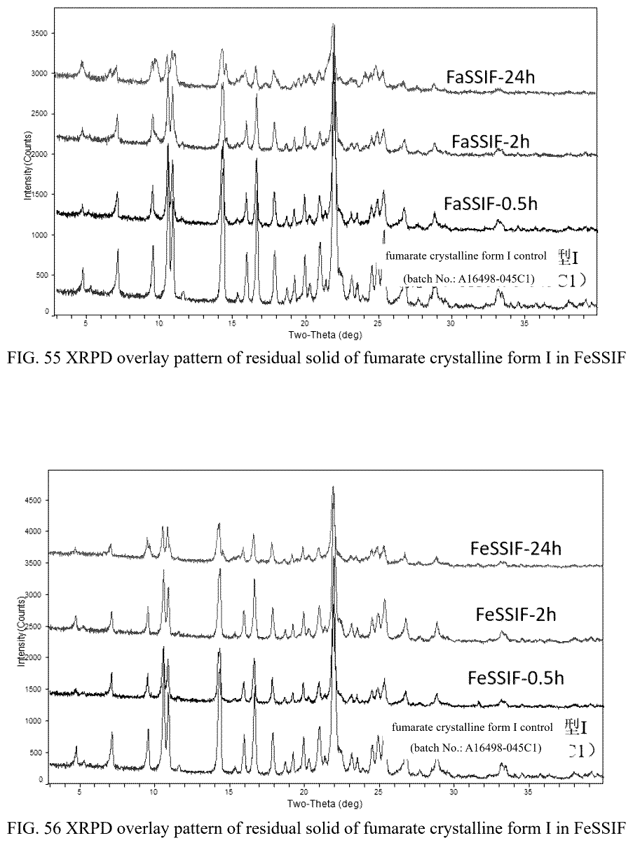

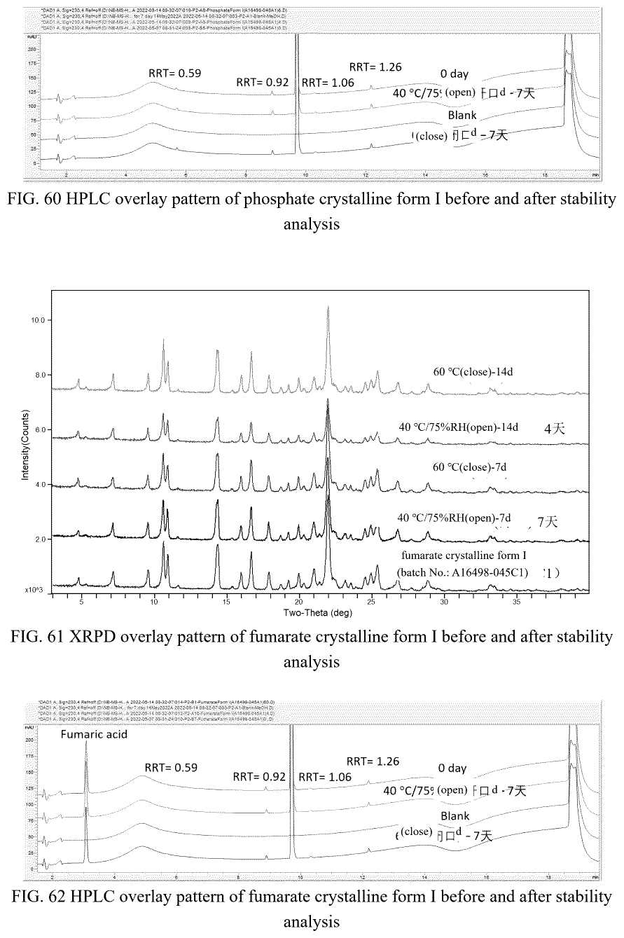

Therefore, phosphate crystalline form I and fumarate crystalline form I are selected as candidate salt forms, solid stability and solubility in biological solvent and water of them and free base crystal form I were studied. Biological solvent solubility tests and stability studies were performed on phosphate crystalline form I and fumarate crystalline form I, and compared with the data of free base crystalline form I, which were shown in Table 18, and FIGS. 33-62.

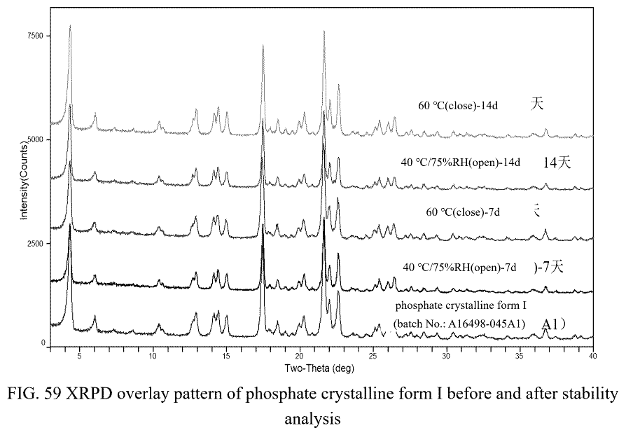

TABLE 18
Comparison of data on free base crystalline form I
and dominant candidate salts

| | | free base | phosphate | fumarate |
|---|---|---|---|---|
| | | crystalline | crystalline | crystalline |
| Items | | form I | form I | form I |
| Solid form | | anhydrous | anhydrous | anhydrous |
| | | crystalline | crystalline form | crystalline |
| | | form | | form |
| Stoichiometric | | N/A | 1:1 | 1: 0.5 |
| ratio | | | | |
| (Base: Acid^{a}) | | | | |
| Crystallinity | | moderate | high | high |
| Polymorphism | | complex, >4 | potentially | simple, 1 |
| | | | complex, 3 | |
| DSC | | 245/247, 64 | 217/221,114 | 252/253, 119 |
| Melting point/ | | | (two overlapping | (m/d) |
| Peak (° C.), | | | endothermic | |
| enthalpy (J/g) | | | peaks, m/d)^{c} | |
| TGA | | 0.2/170-250 | 0.2/RT-50 | 1.0/180-235 |
| Weight loss %/ | | | | |
| temperature (° C.) | | | | |
| DVS | | 0.8, 0.9 | 1.3/1.6 | 0.2/0.2 |
| The weight gains | | slight | slight | almost non |
| by vapor | | hygroscopic | hygroscopic | hygroscopic |
| sorption % | | | | |
| @80/90% RH | | | | |
| Solubility^{b} | Water | <LOD/<LOD/ | <LOD/<LOD/ | <LOD/<LOD/ |
| (mg/mL) | | <LOD | <LOD | <LOD |
| 0.5/2/24 h | SGF | <LOD/<LOD/ | <LOD/<LOD/ | <LOD/<LOD/ |
| | | <LOD | <LOD | <LOD |
| | FaSSIF | 0.014/0.013/ | 0.057/0.048/0.022 | 0.011/0.011/ |
| | | 0.011 | | 0.022 |
| | FeSSIF | 0.181/0.221/ | 1.029/1.175/1.948 | 0.197/0.175/ |
| | | 0.418 | | 0.530 |

| | | | |
|---|---|---|---|
| Solid form | except for | dissociate to free | unchanged |
| of residual | conversion to | base in FeSSIF | crystalline |
| solids in solubility | amorphous | and FaSSIF, | form |
| analysis | in SGF, the | convert to | (See |
| | crystalline form | amorphous | FIGS. 44-56) |
| | is unchanged in | form in SGF | |
| | other media | | |
| Solid state | good physical | good physical and | good physical |
| stability-14 days | and chemical | chemical stability | and chemical |
| 60° C./Closed and | stability | (See FIGS. 59 and | stability |
| 40° C./75% | | 60) | (See FIGS. 61 |
| RH (Open) | | | and 62) |

^{a}Salt formation ratio; a salt formation ratio of inorganic salt was determined by ion chromatography, and salt formation ratio of organic salt was analyzed by ¹H-NMR
^{b}Samples were in duplicate, and data were the mean of experimental data; LOQ = 0.002 mg/mL
^{c}m/d: Melting accompanied with decomposition

### 3.2 Scale-Up Production of Dominant Salt Forms, and Test of Solubility and Stability Thereof

Based on solid state characterization data and production feasibility, phosphate crystalline form I, fumarate crystalline form I and free base crystalline form I were selected for scale-up preparation, and solubility in biological solvent test and solid-state stability research were performed.

### 3.2.1 Scale-Up Preparation of Phosphate Crystalline Form I

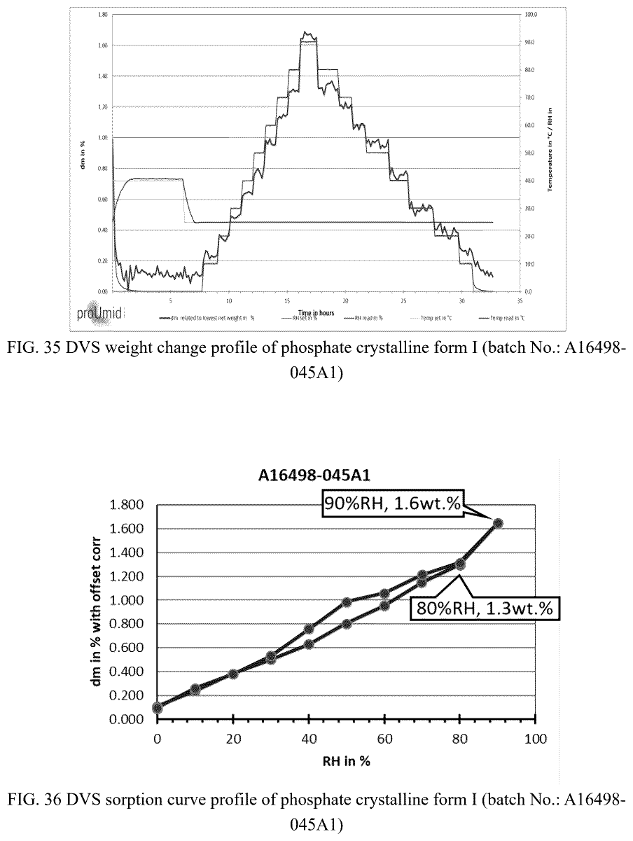

The preparation of phosphate crystalline form I was scaled up to 350 mg, and the experimental details are presented in section 2.8.4. As shown in FIGS. 19 and 33, the resulting solid (batch No.: A16498-045A1) was irregular crystals with agglomeration and high crystallinity, and it was consistent with phosphate crystalline form I. As shown in FIG. 34, two overlapping endothermic peaks can be observed in the DSC curve, which are caused by melting accompanied by decomposition. TGA showed 0.2% weight loss from room temperature to 50° C. No significant solvent residue was detected by ¹H-NMR. The acid-base stoichiometric ratio is 1:1. Thus, phosphate crystalline form I was successfully prepared in a yield of 93%. As shown in FIGS. 35-37, the DVS results showed that phosphate crystalline form I was slightly hygroscopic, the vapor sorption gains were about 1.3% and 1.6% under the condition of 80% RH and 90% RH respectively, and the crystalline form remained unchanged after DVS analysis.

### 3.2.2 Scale-Up Production of Fumarate Crystalline Form I

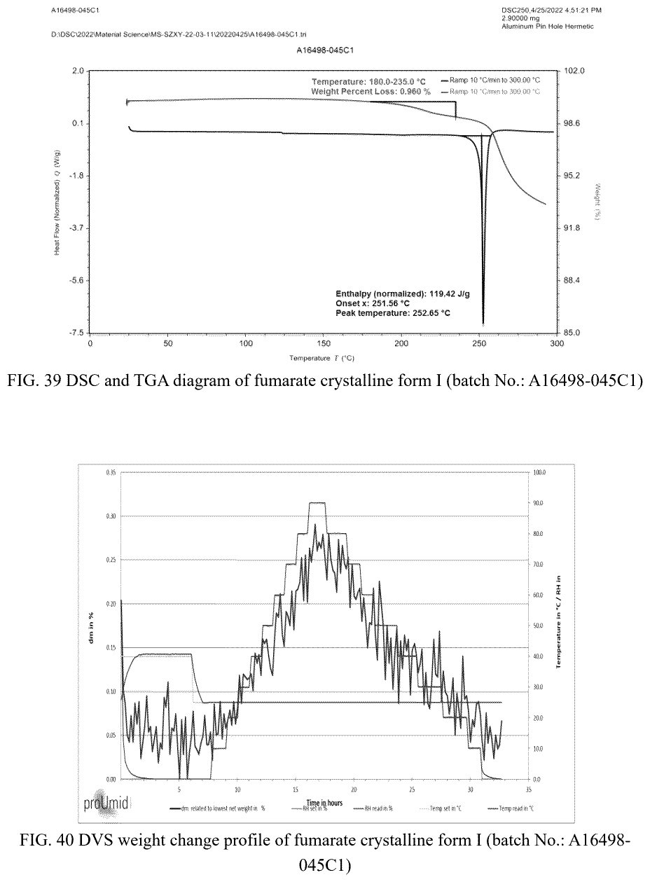

Fumarate crystalline form I (batch No.: A16498-045C1) was prepared on a 350 mg scale up according to the experimental details in section 2.12. As shown in FIGS. 38-42, the crystals had higher crystallinity, consistent with fumarate form I. Fumarate crystalline form I is a microcrystal with a size of less than 5 m. ¹H-NMR confirmed that the stoichiometric ratio was 1:0.5 (base:acid). An endothermic peak was observed in the DSC curve, which was caused by the melting accompanied by decomposition. TGA showed a weight loss of 1.0% over the temperature range 180° C. and 235° C. Thus, fumarate crystalline form I was successfully prepared in 85% yield. As shown in FIGS. 39-41, DVS results indicate that fumarate crystalline form I is almost non-hygroscopic, with vapor sorption gains of about 0.15% and 0.20% at 80% RH and 90% RH, respectively, and the crystalline form remained unchanged after DVS analysis.

### 3.3 Solubility Analysis in Biological Solvents and Water

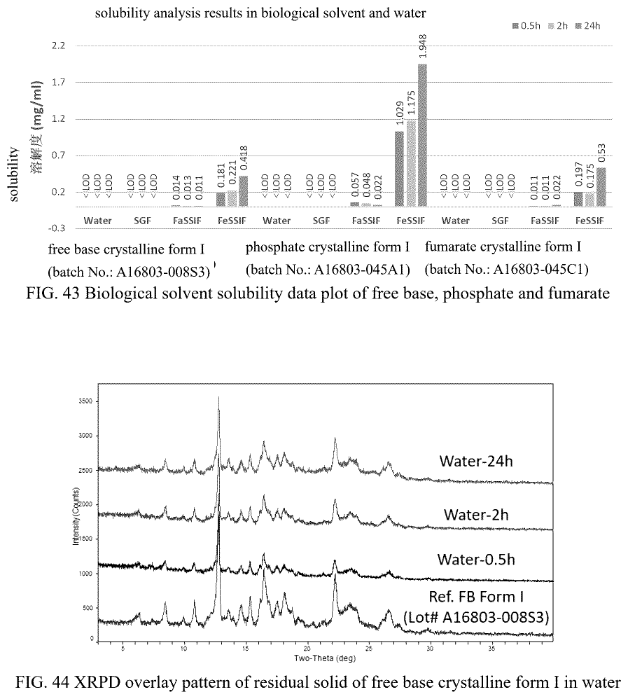

Solubilities of free base crystalline form I (batch No.: A16803-00853), phosphate crystalline form I (batch No.: A16498-045A1) and fumarate crystalline form I (batch No.: A16498-045C1) in biological solvents (SGF, FaSSIF, FeSSIF) and water were tested, the target concentration was set at 5 mg/mL, the temperature was 37° C. The data results were shown in Table 19 and FIGS. 43-56.

Compound A has a poor solubility in SGF and water (<LOD), and has a certain solubility in FeSSIF (0.2-1.9 mg/mL). Solubility in FaSSIF and FeSSIF:phosphate crystalline form I>fumarate crystalline form I≥free base crystalline form I.

As shown in FIGS. 44-47, free base crystalline form I remained unchanged in water, FaSSIF and FeSSIF, but turned amorphous after 2 h in SGF.

As shown in FIGS. 48-52, the XRPD pattern of the residual solid in water of phosphate crystalline form I showed an additional peak at 160 (20). Phosphate crystalline form I became amorphous after shaking for 2 h in SGF. Newform 1 appeared after 0.5 hours shaking in FaSSIF and FeSSIF, and new form 2 was obtained after 24 hours in FaSSIF. Form 1 and Form 2 were unstable and converted to amorphous and form 3, respectively, after drying. The content of PO₄³⁻ in the residual solids were analyzed by IC and the results were shown in Table 20, where phosphate crystalline form I dissociated to free base in FaSSIF and FeSSIF. XRPD showed a moderately high crystallinity for form 3, and no significant organic solvent residue was detected by ¹H-NMR. Multiple endothermic peaks were detected by DSC, TGA showed 0.40 weight loss at 130-180° C., no significant PO₄³⁻ was detected by IC. Thus, it was determined that form 3 may be a new anhydrous crystalline form of the free base.

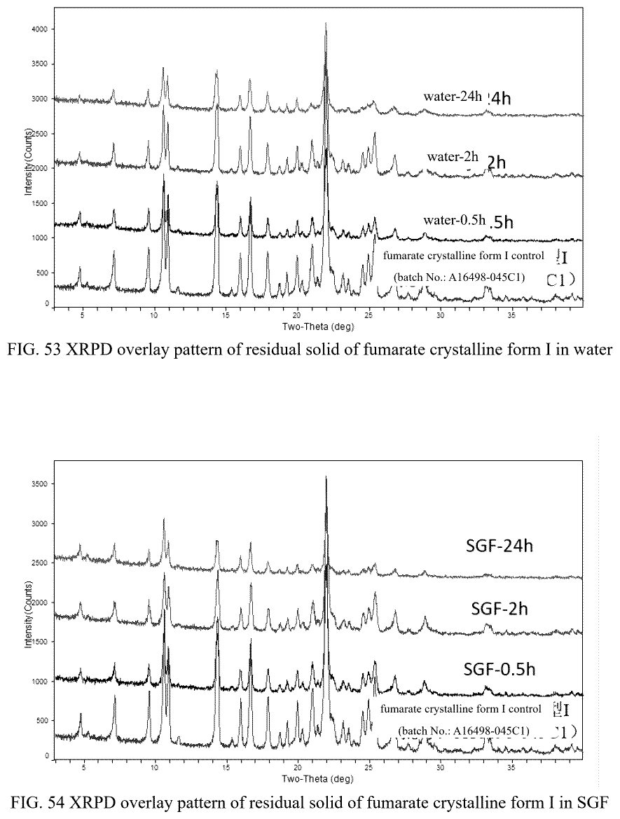

As shown in FIGS. 53-56, fumarate crystalline form I remained unchanged in biological solvents and water.

TABLE 19
Solubility analysis results in biological solvents and water

| | | | Solubility | | | | | | | |
|---|---|---|---|---|---|---|---|---|---|---|
| Sample | | | (mg/mL)^{a} | | | pH | | | | |
| (Batch | | Medium | 0.5 | 2 | 24 | 0.5 | 2 | 24 | XRPD | |
| No.) | Medium | pH | h | h | h | h | h | h | 0.5 h | 24h |
| Free base | water | 6.6 | <LOD | <LOD | <LOD | 6.6 | 6.5 | 6.4 | unchanged | unchanged |
| crystalline | | | | | | | | | | |
| form I | SGF | 1.2 | <LOD | <LOD | <LOD | 1.3 | 1.4 | 1.2 | unchanged | approximate |
| (A16803- | | | | | | | | | | amorphous |
| 008S3) | FaSSIF | 6.5 | 0.014 | 0.013 | 0.011 | 6.4 | 6.3 | 6.4 | unchanged | unchanged |
| | FeSSIF | 5.0 | 0.181 | 0.221 | 0.418 | 5.0 | 5.0 | 5.1 | unchanged | unchanged |
| Phosphate | water | 6.6 | <LOD | <LOD | <LOD | 2.8 | 2.7 | 2.6 | additional peaks at 16° | |
| crystalline | | | | | | | | | (2θ) | |
| form I | SGF | 1.2 | <LOD | <LOD | <LOD | 1.2 | 1.4 | 1.2 | approximate | amorphous |
| (A16498- | | | | | | | | | amorphous | |
| 045A1) | FaSSIF^{b} | 6.5 | 0.057 | 0.048 | 0.022 | 5.7 | 5.5 | 5.5 | Form 1 | Form 2 |
| | FeSSIF | 5.0 | 1.029 | 1.175 | 1.948 | 4.9 | 4.9 | 5.0 | Form 1 | Form 1 |
| Fumarate | water | 6.6 | <LOD | <LOD | <LOD | 4.6 | 4.3 | 4.2 | unchanged | unchanged |
| crystalline | | | | | | | | | | |
| form I | SGF | 1.2 | <LOD | <LOD | <LOD | 1.3 | 1.4 | 1.3 | unchanged | unchanged |
| (A16498- | FaSSIF | 6.5 | 0.011 | 0.011 | 0.022 | 6.3 | 6.2 | 6.0 | unchanged | unchanged |
| 045C1) | FeSSIF | 5.0 | 0.197 | 0.175 | 0.530 | 5.0 | 5.0 | 5.0 | unchanged | unchanged |

^{a}Samples were in duplicate, and data were the mean of experimental data; LOQ = 0.002 mg/mL;
^{b}Form 1 and form 2 were unstable and converted to amorphous and form 3, respectively, after drying

TABLE 20
IC results of residual solid of phosphate crystalline form I

| residual solid | | XPRD Results | | | Dissociation |
|---|---|---|---|---|---|
| | point | Wet | Dry | PO₄³⁻ | degree |
| medium | of time | sample | sample | (eq.) | (%) |
| FaSSIF | 0.5 h | Form 1 | Amorphous | 0.1 | 91 |
| | 2 h | Form 1 | Amorphous | 0.03 | 97 |
| | 24 h | Form 2 | Form 3 | <0.006 | >99 |
| FeSSIF | 0.5 h | Form 1 | Amorphous | <0.006 | >99 |
| | 24 h | Form 1 | Amorphous | 0 | 100 |

### 3.4 Research on Solid State Stability

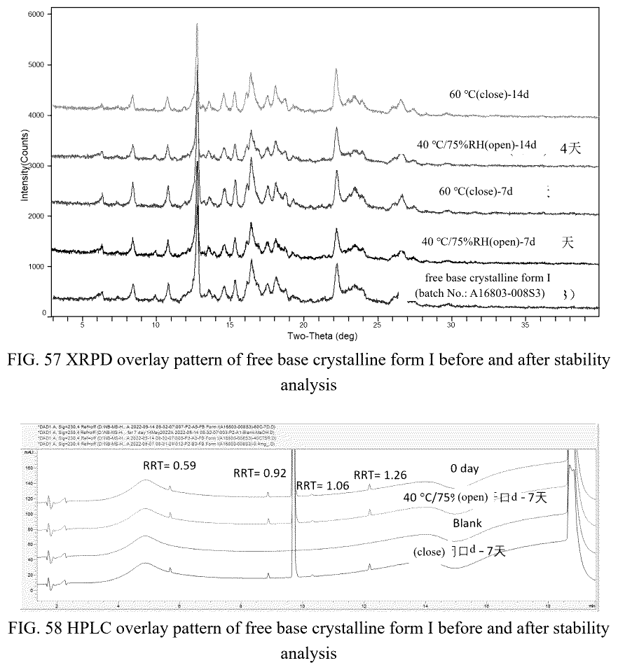

Free base crystalline form I, phosphate crystalline form I and fumarate crystalline form I were placed in an environment of 60° C. (closed) and 40° C./75 RH (open) for 14 days, XRPD analysis and purity analysis were performed at various points of time, and solid-state stability was evaluated. The results were shown in Table 21 and FIGS. 57-62, and free base crystalline form I, phosphate crystalline form I and fumarate crystalline form I are physically and chemically stable under the test conditions described above.

TABLE 21
Evaluation results of stability

| Sample | | Purity | |
|---|---|---|---|
| (Batch No.) | conditions | (Area %) | XRPD |
| Free base | 0 d | 98.63 | N/A |
| crystalline form I | 7 d-40° C./75% RH | 98.60 | unchanged |
| (A16803-008S3) | 7 d-60° C./capped | 98.61 | unchanged |
| | 14 d-40° C./75% RH | 98.61 | unchanged |
| | 14 d-60° C./capped | 98.70 | unchanged |
| Phosphate | 0 d | 98.78 | N/A |
| crystalline form I | 7 d-40° C./75% RH | 98.78 | unchanged |
| (A16498-045A1) | 7 d-60° C./capped | 98.78 | unchanged |
| | 14 d-40° C./75% RH | 98.78 | unchanged |
| | 14 d-60° C./capped | 98.78 | unchanged |
| Fumarate | 0 d | 99.14 | N/A |
| crystalline form I | 7 d-40° C./75% RH | 99.13 | unchanged |
| (A16498-045C1) | 7 d-60° C./capped | 99.14 | unchanged |
| | 14 d-40° C./75% RH | 99.13 | unchanged |
| | 14 d-60° C./capped | 99.13 | unchanged |

### 4. Related Properties of Compound A Fumarate

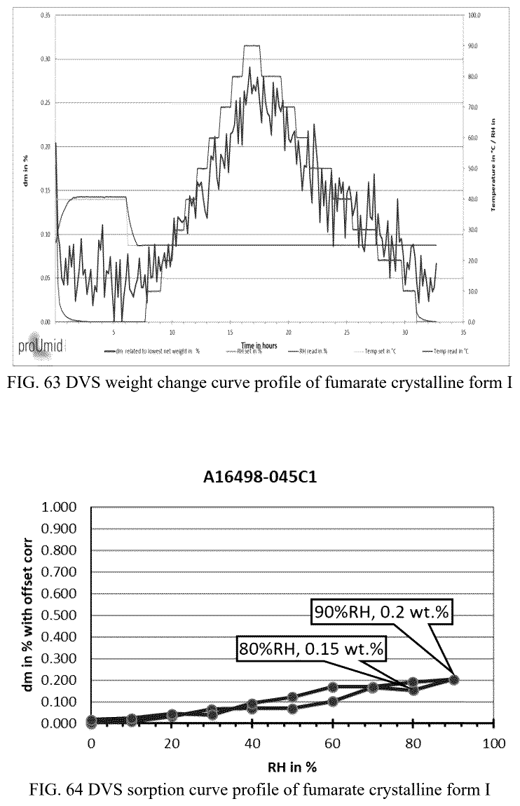

A fumarate crystalline form was obtained by salt formation of free base and 1.1 equivalents of fumaric acid, named fumarate crystalline form I. (The synthesis is presented in section 2.12, batch No.: A16498-067-B1). Fumarate crystalline form I was characterized by: XRPD characteristic peaks at 2θ: 4.703°, 5.234°, 7.100°, 9.515°, 10.561°, 10.864°, 11.566°, 14.336°, 15.324°, 15.926°, 16.625°, 17.834°, 18.671°, 19.193°, 19.904°, 20.972°, 21.320°, 21.607°, 21.908°, 22.058°, 23.118°, 23.487°, 24.089°, 24.899°, 25.162°, 25.337°, 26.463°, 26.767°, 28.444°, 28.809°, 29.017°, 29.496°, 30.621°, 31.059°, 32.281°, 32.640°, 33.214°, 33.407°, 34.009°, 34.480°, 35.672°, 37.870°, 38.210°, 39.076°, 39.488°; ¹H-NMR shows that the molar ratio of acid to base is 0.5:1; DSC shows an endothermic peak at 251° C., which is a melting point, and TGA result shows no obvious weight loss below 200° C., thus being an anhydrous compound; substantially non-hygroscopic; it was physicochemically stable for 7 days at 60° C./closed and 40° C./75% RH. See FIGS. 25 to 28, and FIGS. 61 to 64.

### 4.1 Research on Solid Storage Stability

### 4.1.1 Solid Stability at 60° C./Closed and at 40° C./75% RH

The solid stability of fumarate crystalline form I was evaluated at 60° C./closed mouth and 40° C./75% RH for 7 days. No change in crystalline form was observed and the chemical purity remained unchanged. Thus, form I was physicochemically stable under these conditions for 7 days. The experimental results were shown in FIGS. 61 and 62.

### 4.1.2 Mechanical Grinding

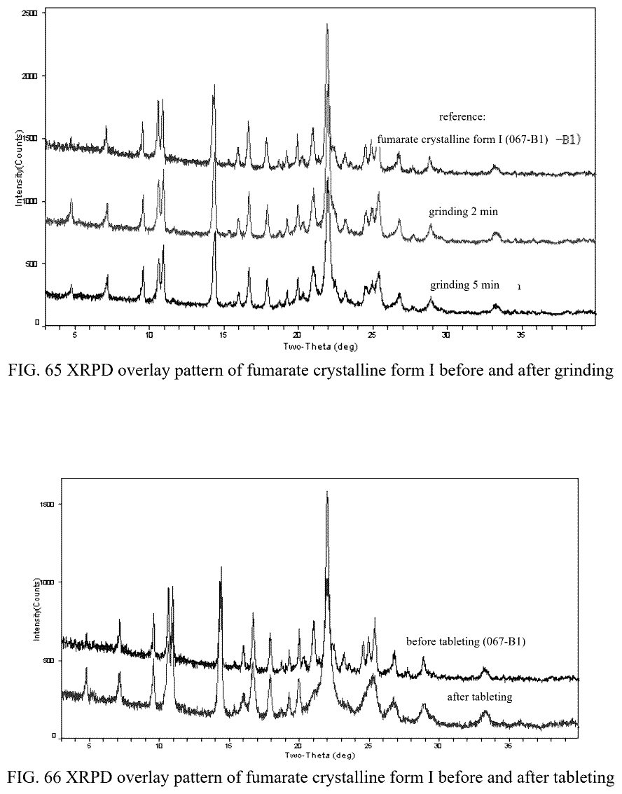

An appropriate amount of form I fumarate crystalline form I (batch No. A16498-067-B1) was ground in a mortar for 5 minutes, with samples taken at 2 and 5 minutes for XRPD. The crystal form is not changed after grinding, the crystallinity was not obviously reduced, and the experimental result was shown in FIG. 65.

### 4.1.3 Research on Tableting

An appropriate amount of fumarate crystalline form I (batch No. A16498-067-B1) was weighed into a mold and compressed manually (40 Mpa, 1 min) followed by XRPD analysis. The crystalline form is unchanged after tableting, the crystallinity was slightly reduced, and the experimental result was shown in FIG. 66.

### 4.2 Polymorph Screening

### 4.2.1 Roughly Measured Solubility

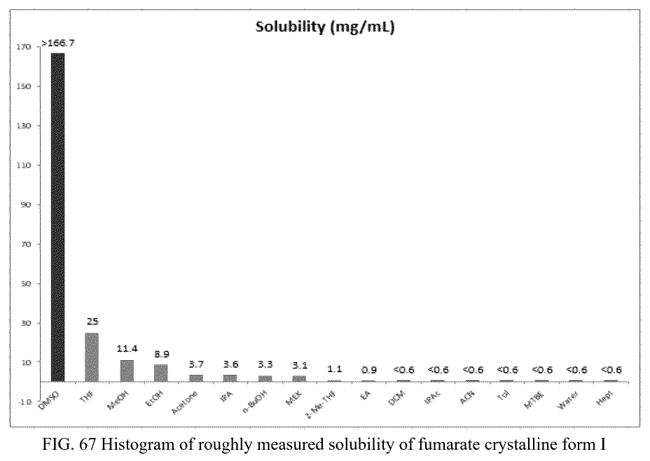

5 mg of fumarate crystalline form I (batch No. A16498-067-B1) were weighed into sample bottles at room temperature and the solvent was added dropwise and stepwise until the drug was dissolved to give a clear solution or reach 1600V, roughly calculating solubility (mg/mL). The results were summarized in Table 22 and FIG. 67. Fumarate crystalline form I has high solubility in DMSO (>167 mg/mL) and very poor solubility in water and most of other organic solvents (<5.0 mg/mL).

TABLE 22
Rough measurement of solubility
of fumarate crystalline form I

| | | Solubility |
|---|---|---|
| No. | Solvent | (mg/mL) |
| 1 | MeOH | 11.4 |
| 2 | EtOH | 8.9 |
| 3 | IPA | 3.6 |
| 4 | n-BuOH | 3.3 |
| 5 | EA | 0.9 |
| 6 | IPAc | <0.6 |
| 7 | acetone | 3.7 |
| 8 | MEK | 3.1 |
| 9 | ACN | <0.6 |
| 10 | THF | 25.0 |
| 11 | 2-Me-THF | 1.1 |
| 12 | DCM | <0.6 |
| 13 | MTBE | <0.6 |
| 14 | Tol | <0.6 |
| 15 | DMSO | >166.7 |
| 16 | water | <0.6 |
| 17 | Hept | <0.6 |

1. The values were rounded to the nearest integer, labeled “<” if not dissolved and “>” if dissolved.

### 4.2.2 Evaporating Crystallization

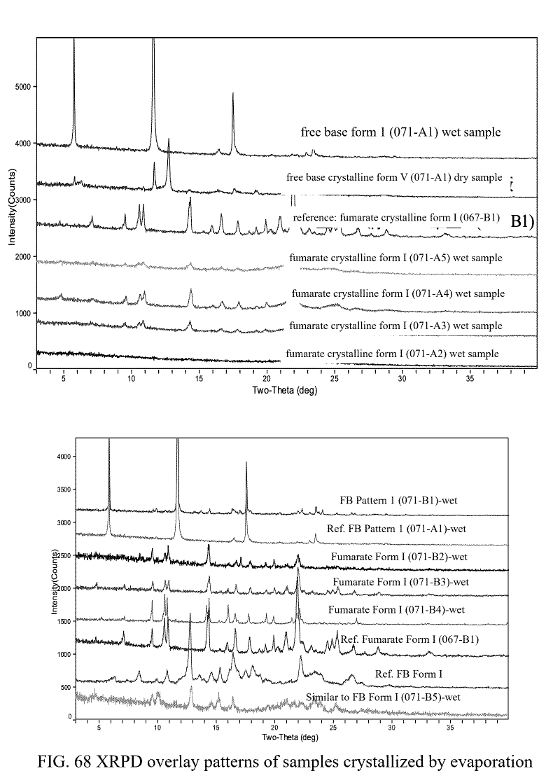

10 mg of fumarate crystalline form I (batch No. A16498-067-1B1) was dissolved in a solvent (1.0 mL) at room temperature or 50° C. to form a clear solution. The solution was equally divided into two parts, one part was dried by blowing with nitrogen gas for quick volatilization, the other part was subjected to slow evaporation at room temperature covered with a thin and perforated film, and no new crystalline form was obtained. However, two new free base crystalline forms were obtained, named free base crystalline form 1 and free base crystalline form V The results were shown in Table 23 and FIG. 68. Free base form 1 may be metastable and converted to free base crystalline form V upon drying. However, no new crystalline form of compound A fumarate was formed.

TABLE 23
Experimental results of evaporating crystallization

| | | | | | | Results |
|---|---|---|---|---|---|---|
| | | | Results | | | (quick |
| | | Batch | (quick volatilization −1 h) | | Batch | volatilization- |
| | Concentration | No. | | Dry | No. | 1d) |
| Solvent | (mg/mL) | A16498 | Wet sample | sample | A16498 | Wet sample |
| MeOH | saturated | 071-A1 | free base form 1 | free base | 071-B1 | free base form |
| | solution | | | crystalline | | 1 |
| | (50° C. to | | | form V | | |
| EtOH | room | 071-A2 | amorphous | / | 071-B2 | fumarate |
| | temperature) | | | | | crystalline |
| | | | | | | form I |
| Acetone | | 071-A3 | fumarate | / | 071-B3 | fumarate |
| | | | crystalline form I | | | crystalline |
| | | | | | | form I |
| MEK | | 071-A4 | fumarate | / | 071-B4 | fumarate |
| | | | crystalline form I | | | crystalline |
| | | | | | | form I |
| THF | 25.0 | 071-A5 | fumarate | / | 071-B5 | similar to free |
| | (room | | crystalline form I | | | base |
| | temperature) | | | | | crystalline |
| | | | | | | form I |

### 4.2.3 Cooling Crystallization

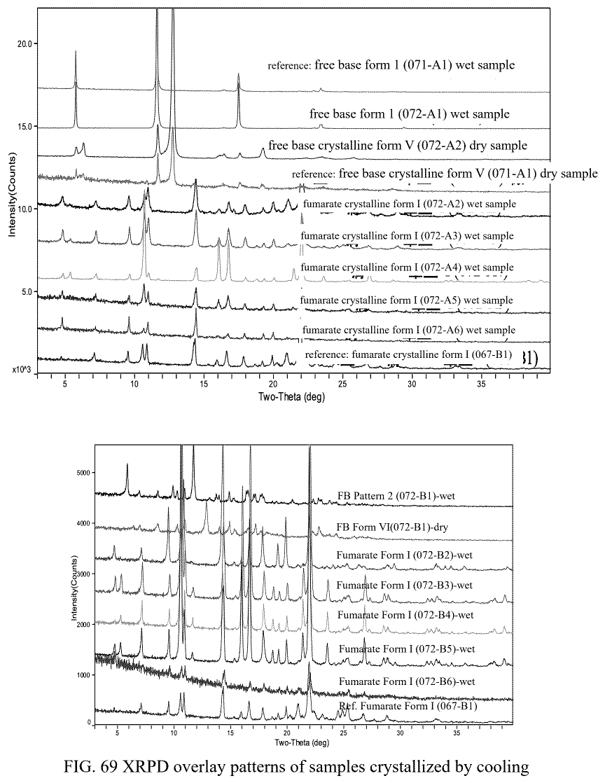

30 mg of fumarate crystalline form I (batch No. A26498-067-B1) was weighed and took into a sample bottle, a solvent was added under 50° C. followed by stirring for 30 minutes. It was filtered through a microfilm into clear vials. The solution was divided into two equal parts, one part of filtrate was placed in a refrigerator (5° C.) for rapid cooling, and the other part of filtrate was placed in a heating metal module with a power off and naturally cooled to room temperature. If the solid precipitated, corresponding characterization was performed, but no new crystalline form was obtained. In a solution in methanol, fumarate crystalline form I dissociated into free base form 1 by rapid cooling and into free base form 2 by slow cooling. Form 1 and form 2 were converted to free base form V and free base form VI, respectively, after drying. The results were shown in Table 24 and FIG. 69. However, no new crystalline form of compound A fumarate was formed.

TABLE 24
Experimental results of cooling crystallization

| | | | | Results | | | Results | |
|---|---|---|---|---|---|---|---|---|
| | | | | (rapid cooling) | | | (slow cooling) | |
| | | | Batch | −2 h | | Batch | −2 h~3 d | |
| Solvent | Volume | Concentration | No. | Wet | Dry | No. | Wet | Dry |
| (v/v) | (mL) | (mg/mL) | A16498 | sample | sample | A16498 | sample | sample |
| MeOH | 1.0 | saturated | 072- | free base | Free | 072- | free base | free base |
| | | | A1 | form 1 | base | B1 | form 2 | crystalline |
| | | | | | crystalline | | | form |
| | | | | | form V | | | VI |
| EtOH | 1.0 | saturated | 072- | fumarate | / | 072- | fumarate | / |
| | | | A2 | crystalline | | B2 | crystalline | |
| | | | | form I | | | form I | |
| Acetone/ | 0.85 | saturated | 072- | fumarate | / | 072- | fumarate | / |
| MeOH | | | A3 | crystalline | | B3 | crystalline | |
| (3/1) | | | | form I | | | form I | |
| DCM/ | 0.85 | 35.3 | 072- | fumarate | / | 072- | fumarate | / |
| MeOH | | | A4 | crystalline | | B4 | crystalline | |
| (3/1) | | | | form I | | | form I | |
| EA/ | 1.0 | saturated | 072- | fumarate | / | 072- | fumarate | / |
| MeOH | | | A5 | crystalline | | B5 | crystalline | |
| (3/1) | | | | form I | | | form I | |
| THF | 0.6 | 58.9 | 072- | fumarate | / | 072- | fumarate | / |
| | | | A6 | crystalline | | B6 | crystalline | |
| | | | | form I | | | form I | |
| | | | | (3 d) | | | | |

### 4.2.4 Antisolvent Precipitation

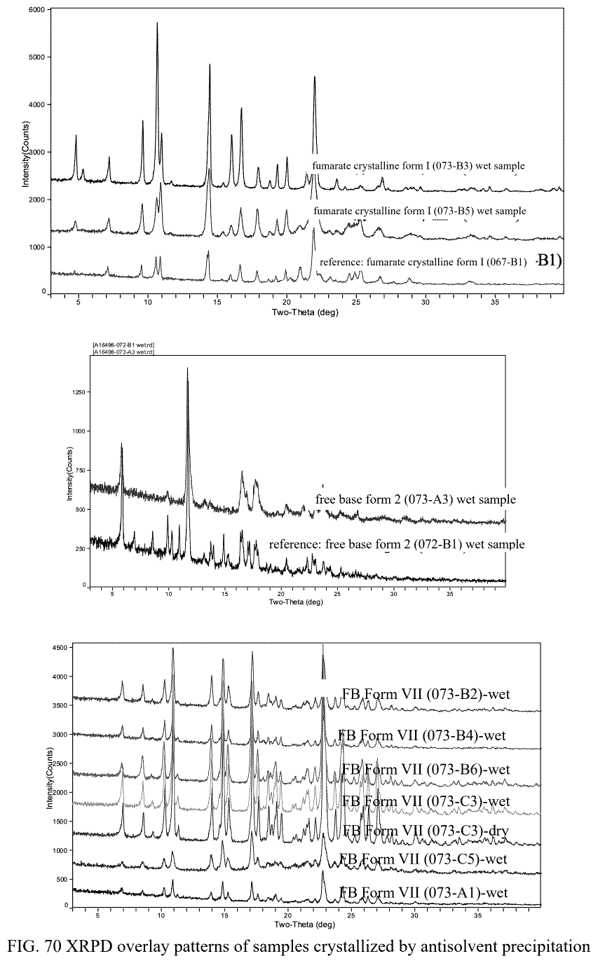

An appropriate amount of fumarate crystalline form I (batch A16498-067-1B1) was dissolved in 0.2-0.4 mL of MeOH, THF or DMSO at room temperature and the solution was filtered to give a clear solution. An anti-solvent was gradually added thereto until a solid precipitated or a solvent volume of was added. If no precipitation occurred, the solution was cooled to 5° C. and stirred for 40 h, then filtered and the filter cake was identified by XRPD analysis and no new crystalline form was obtained. However, a new free base crystalline form was obtained, named as free base crystalline form VII. The results were shown in Table 25 and FIG. 70. However, no new crystalline form of compound A fumarate was formed.

TABLE 25
Experimental results of Antisolvent precipitation

| Batch | | | | Results | |
|---|---|---|---|---|---|
| No. | | Anti- | | Wet | Dry |
| A16498 | Solvent | solvent | Vₛₒₗᵥₑₙₜ/Vₐₙₜᵢ₋ₛₒₗᵥₑₙₜ | sample | sample |
| 073-A1 | MeOH | ACN | 1/10 | free base crystalline | / |
| | (Saturated, | | | form VII | |
| 073-A2 | 0.3 mL) | MTBE | 1/10 | clear solution | / |
| 073-A3 | | H₂O | 1/0.3 | similar to free base | / |
| | | | | form 2 | |
| 073-B1 | THF | EA | 1/10 | clear solution | / |
| 073-B2 | (25 mg/mL, | IPAc | 1/10 | free base crystalline | / |
| | 0.4 mL) | | | form VII | |
| 073-B3 | | ACN | 1/10 | fumarate crystalline | / |
| | | | | form I | |
| 073-B4 | | MTBE | 1/10 | free base crystalline | / |
| | | | | form VII | |
| 073-B5 | | Hept | 1/2.5 | fumarate crystalline | / |
| | | | | form I | |
| 073-B6 | | H₂O | 1/1.25 | free base crystalline | / |
| | | | | form VII | |
| 073-C1 | DMSO | EA | 1/10 | clear solution | / |
| 073-C2 | (100 mg/mL, | IPAC | 1/10 | clear solution | / |
| 073-C3 | 0.2 mL) | ACN | 1/10 | free base crystalline | free base |
| | | | | form VII | crystalline |
| | | | | | form VII |
| 073-C4 | | MTBE | 1/10 | clear solution | / |
| 073-C5 | | H₂O | 1/0.35 | free base crystalline | / |
| | | | | form VII | |

### 4.2.5 Research on Trituration

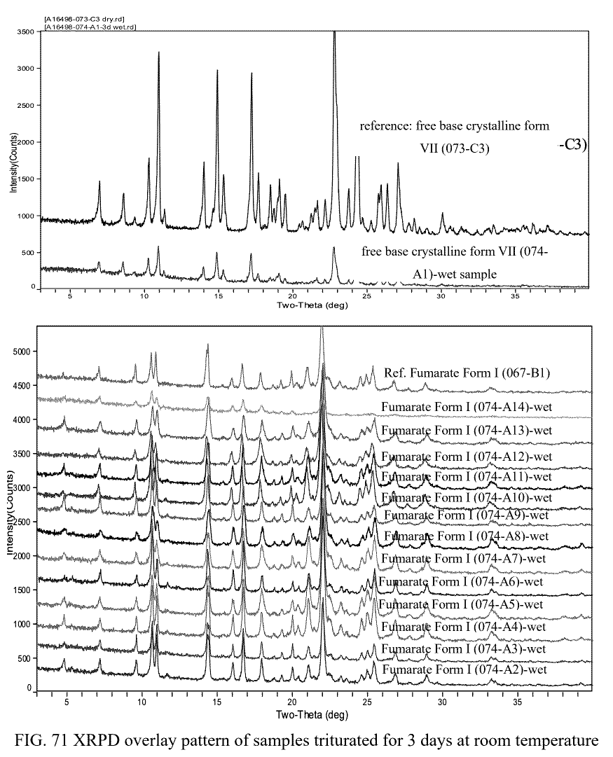

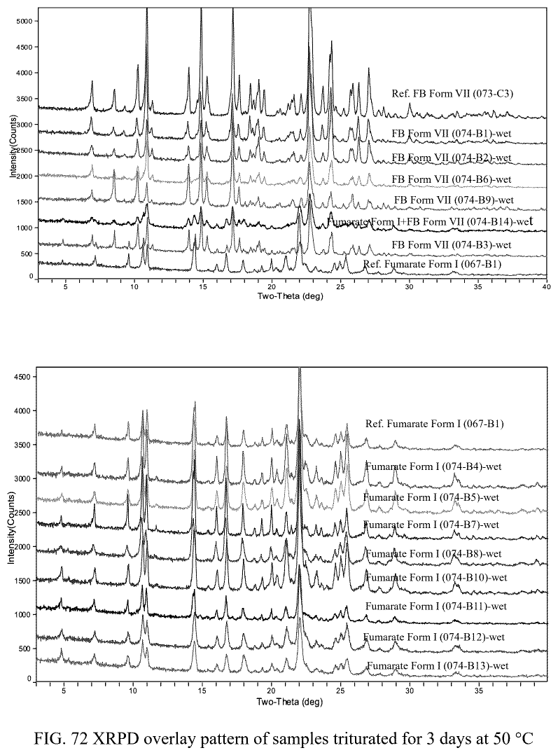

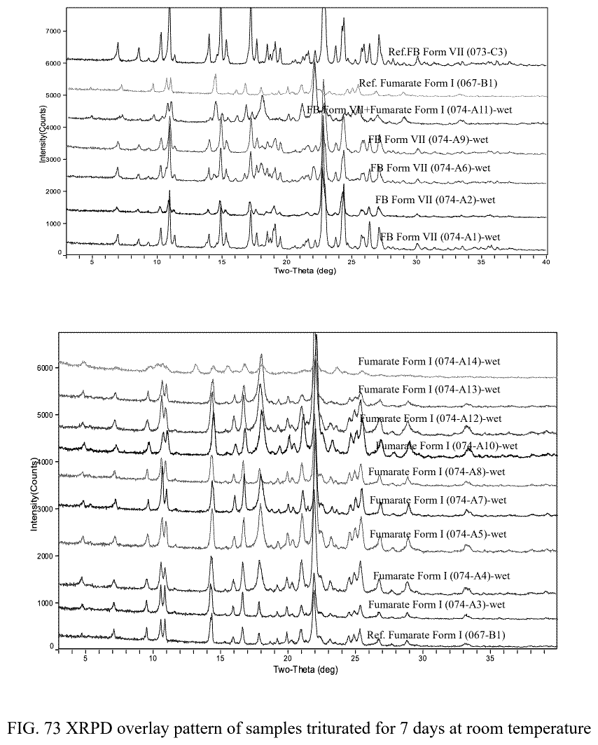

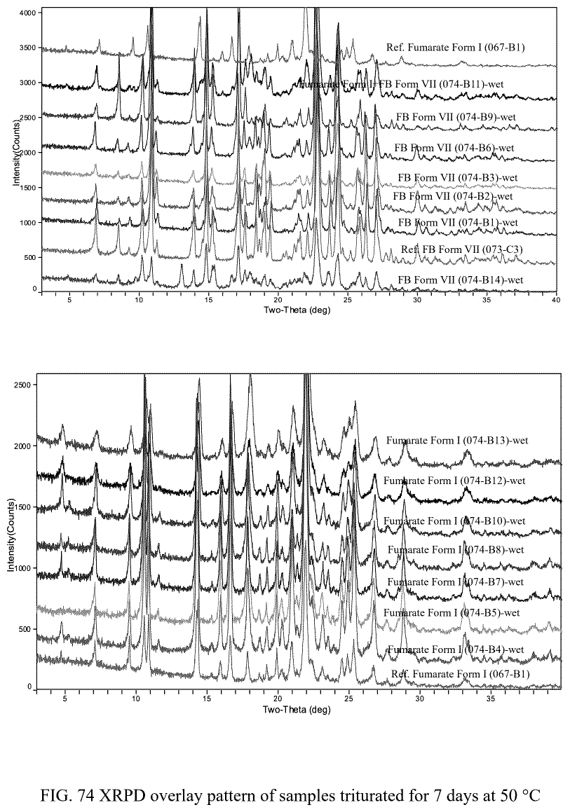

30 mg of fumarate crystalline form I (batch A16498-067-B31) was weighed and taken into a sample bottle, a solvent was added to form a suspension with a concentration of 30 mg/mL, and which was stirred at room temperature or 50° C. for 3 days or 7 days. The mixture was filtered to collect solid, and the filter cake was identified by XRPD, no new fumarate crystalline form was formed. Fumarate crystalline form I dissociated into free base crystalline form VII in alcohol, water, 2-Me-THF, MTBE and acetone. The results were shown in Tables 26-27 and FIGS. 71-74. However, no new crystalline form of compound A fumarate was formed.

TABLE 26
Experimental results of triturating for 3 days

| | Room temperature for 3 d | | 50° C. for 3 d | |
|---|---|---|---|---|
| | Batch | | Batch | |
| | No. | | No. | |
| Solvent | A16498 | XRPD | A16498 | XRPD |
| EtOH | 074-A1 | free base crystalline | 074-B1 | free base crystalline |
| | | form VII | | form VII |
| IPA | 074-A2 | fumarate crystalline | 074-B2 | free base crystalline |
| | | form I | | form VII |
| n-BuOH | 074-A3 | fumarate crystalline | 074-B3 | free base crystalline |
| | | form I | | form VII + fumarate |
| | | | | crystalline form I |
| EA | 074-A4 | fumarate crystalline | 074-B4 | Fumarate crystalline |
| | | form I | | form I |
| IPAc | 074-A5 | fumarate crystalline | 074-B5 | Fumarate crystalline |
| | | form I | | form I |
| Acetone | 074-A6 | fumarate crystalline | 074-B6 | free base crystalline |
| | | form I | | form VII |
| MEK | 074-A7 | fumarate crystalline | 074-B7 | fumarate crystalline |
| | | form I | | form I |
| ACN | 074-A8 | fumarate crystalline | 074-B8 | fumarate crystalline |
| | | form I | | form I |
| 2-Me-THF | 074-A9 | fumarate crystalline | 074-B9 | free base crystalline |
| | | form I | | form VII |
| DCM | 074-A10 | fumarate crystalline | 074-B10 | Fumarate crystalline |
| | | form I | | form I |
| MTBE | 074-A11 | fumarate crystalline | 074-B11 | Fumarate crystalline |
| | | form I | | form I |
| Tol | 074-A12 | fumarate crystalline | 074-B12 | Fumarate crystalline |
| | | form I | | form I |
| Hept | 074-A13 | fumarate crystalline | 074-B13 | Fumarate crystalline |
| | | form I | | form I |
| H₂O | 074-A14 | fumarate crystalline | 074-B14 | free base crystalline |
| | | form I | | form VII + fumarate |
| | | | | crystalline form I |

TABLE 27
Experimental results of triturating for 7 days

| | Room temperature for 7 d | | 50° C. for 7 d | |
|---|---|---|---|---|
| | Batch No. | | Lot# | Batch No. |
| Solvent | A16498 | XRPD | A16498 | A16498 |
| EtOH | 074-A1 | free base crystalline | 074-B1 | free base crystalline |
| | | form VII | | form VII |
| IPA | 074-A2 | free base crystalline | 074-B2 | free base crystalline |
| | | form VII | | form VII |
| n-BuOH | 074-A3 | fumarate crystalline | 074-B3 | free base crystalline |
| | | form I | | form VII |
| EA | 074-A4 | fumarate crystalline | 074-B4 | fumarate crystalline |
| | | form I | | form I |
| IPAc | 074-A5 | fumarate crystalline | 074-B5 | fumarate crystalline |
| | | form I | | form I |
| Acetone | 074-A6 | free base crystalline | 074-B6 | free base crystalline |
| | | form VII | | form VII |
| MEK | 074-A7 | fumarate crystalline | 074-B7 | fumarate crystalline |
| | | form I | | form I |
| ACN | 074-A8 | fumarate crystalline | 074-B8 | fumarate crystalline |
| | | form I | | form I |
| 2-Me-THF | 074-A9 | fumarate crystalline | 074-B9 | free base crystalline |
| | | form I | | form VII |
| DCM | 074-A10 | fumarate crystalline | 074-B10 | fumarate crystalline |
| | | form I | | form I |
| MTBE | 074-A11 | free base crystalline | 074-B11 | free base crystalline |
| | | form VII + fumarate | | form VII + fumarate |
| | | crystalline form I | | crystalline form I |
| Tol | 074-A12 | fumarate crystalline | 074-B12 | fumarate crystalline |
| | | form I | | form I |
| Hept | 074-A13 | fumarate crystalline | 074-B13 | fumarate crystalline |
| | | form I | | form I |
| H₂O | 074-A14 | fumarate crystalline | 074-B14 | free base crystalline |
| | | form I + multiple | | form VII + multiple |
| | | peaks | | peaks |

In conclusion, various methods were used to screen polymorphism by using fumarate crystalline form I as a raw material, including volatilization crystallization, trituration, anti-solvent precipitation, cooling crystallization, mechanical grinding and the like, no new fumarate crystalline form of compound A was formed. This indicated that compound A fumarate crystalline form I is very likely to be the only crystalline form of compound A fumarate. In summary, fumarate crystalline form I exhibited acceptable properties, it is suitable for further development.

### 5 Analysis Methods

### 5.1 Polarization Microscope (PLM)

The PLM analysis was performed on a polarizing microscope with instrument model ECLIPSE LV100POL (Nikon, Japan). A small amount of sample was placed on a glass slide, pine tar was added dropwise to disperse the sample, it was covered with another cover slide, and observation and photographing were performed with a 4-20× objective lens.

### 5.2 X-Ray Powder Diffraction (XRPD)

The solid sample was analyzed by using an X-ray powder diffractometer. The sample was placed on a zero-background silicon wafer and pressed flat gently. The X-ray powder diffractometer parameters were shown in Table 28.

TABLE 28
Parameters of X-ray powder diffractometer

| | |
|---|---|
| Instrument | PANalytical, Empyrean |
| Ray | Cu Kα (λ = 1.5418 Å) |
| Detector | PIXcel^{1D} |
| Scaning angle | 3-40° (2θ) |
| Scanning step | 0.013° (2θ) |
| Scanning rate | 20.4 seconds/step |
| Light tube | 45 kV/40 mA |
| voltage/current | |
| Divergence slit | 1/8° |
| Rotate | open |
| Sample pan | zero background pan |

### 5.3 Thermogravimetric Analysis (TGA)

The model of the thermogravimetric analyzer is TGA 55 (TA Instruments, US). 1-5 mg of the sample was placed in a peeled aluminum sample pan and the temperature program was as shown in Table 29. The data was analyzed by using TRIOS software.

TABLE 29
Parameters of TGA

| | |
|---|---|
| Instrument | TA, Discovery TGA 55 |
| Sample pan | Aluminum pan with a perforated cover |
| Temperature range | RT-300 ° C. |
| Heating rate | 10° C./min |
| Purge gas | N₂ |
| Gas flow rate | balancing chamber: 40 mL/min |
| | Sample room 60 mL/min |

### 5.4 Differential Scanning Calorimetry (DSC)

The model of the differential scanning calorimetry is DSC 250 (TA Instruments, US). 1-3 mg of the sample was placed in a perforated DSC sample pan, heat test was conducted according to the parameters in Table 30 and the data was analyzed by using TRIOS software.

TABLE 30
Parameters of DSC test method

| | |
|---|---|
| Instrument | TA, Discovery DSC 250 |
| Sample pan | Aluminum pan with a perforated cover |
| Temperature range | 25-300° C. |
| Heating rate | 10° C./min |
| Purge gas | N₂ |
| Gas flow rate | 50 mL/min |

### 5.5 Dynamic Vapor Sorption Instrument (DVS)

The water vapor sorption/desorption data of the samples were collected on a dynamic vapor sorption instrument (proUmid GmbH & Co. KG, Germany). Approximately 50-70 mg of sample was placed in a sample pan previously tared and the weight of the sample was measured accurately. The test parameters for the anhydrous crystalline form were shown in Table 31.

TABLE 31
Parameters of DVS analysis for anhydrous crystalline form

| Instrument | ProUmid GmbH & Co. KG | |
|---|---|---|
| Test temperature | 25° C. | |
| Test interval | 10 min | |
| Minimum balance time | 50 min | |
| Maximum balance time | 120 min | |
| Weight limit | 100% | |
| Balance condition | 0.01%/45 min | |
| Atmosphere circulation #1 | 0%-0% | 40° C.; 1 step; each step |
| | | 3 h |
| Atmosphere circulation #2 | 0%-90% | 25° C.; 9 steps |
| Atmosphere circulation #3 | 80%-0% | 25° C.; 8 steps |

| | |
|---|---|
| Sorption humidity % RH | 0, 10, 20, 30, 40, 50, 60, 70, 80, 90 |
| Desorption humidity % RH | 80, 70, 60, 50, 40, 30, 20, 10, 0 |

### 5.6 Hydrogen Nuclear Magnetic Resonance (¹H-NMR)

The hydrogen spectrum information of the sample was collected by using a Bruker 400 MHz instrument. The sample was prepared in DMSO-d6 solvent and tested by using the parameters in Table 32. Data were analyzed by using MestReNova.

TABLE 32
Parameters of ¹H-NMR analysis

| | |
|---|---|
| Instrument | Bruker |
| Frequency | 400 mHz |
| Scan number of times | 16-32 |
| Temperature | 295K |
| Relaxation delay | 1 s |

### 5.7 High Performance Liquid Chromatography (HPLC)

HPLC analysis was performed by using an Agilent HPLC 1260 series instrument and the HPLC methods used for solubility and stability determinations were shown in Tables 33 and 34, respectively.

TABLE 33
Method of HPLC analysis for solubility test

| Instrument information | Agilent 1260 series | | |
|---|---|---|---|
| Chromatography column | XBridge C18 4.6*150 mm 3.5 μm | | |
| Column temperature | 35° C. | | |
| Mobile phase | A: 0.05% TFA in H₂O | | |
| | B: 0.05% TFA in ACN | | |
| Flow rate | 1.0 mL/min | | |
| Injection volume | 5 μL | | |
| Detector | DAD | | |
| Wavelength | 230 nm | | |
| Run time | 15.1 min | | |
| Post run time | 3.0 min | | |
| Diluent | methanol | | |
| | Time (min) | % A | % B |
| Gradient | 0.0 | 90 | 10 |
| | 4.0 | 40 | 60 |
| | 6.0 | 30 | 70 |
| | 7.0 | 30 | 70 |
| | 10.0 | 10 | 90 |
| | 15.0 | 10 | 90 |
| | 15.1 | 90 | 10 |

TABLE 34
Method of HPLC analysis for stability test

| Instrument information | Agilent 1260 series | | |
|---|---|---|---|
| Chromatography column | Waters Sunfire 4.6*150 mm 3.5 μm | | |
| Column temperature | 40° C. | | |
| Mobile phase | A: 0.05% TFA in H₂O | | |
| | B: 0.05% TFA in ACN | | |
| Flow rate | 1.0 mL/min | | |
| Injection volume | 5 μL | | |
| Detector | DAD | | |
| Wavelength | 230 nm | | |
| Run time | 20.0 min | | |
| Diluent | methanol | | |
| | Time (min) | % A | % B |
| Gradient | 0.0 | 95 | 5 |
| | 12.0 | 5 | 95 |
| | 16.0 | 5 | 95 |
| | 16.1 | 95 | 5 |
| | 20 | 95 | 5 |

### 5.8 Ion Chromatography (IC)

IC analysis of the salts was performed by using a Thermo ICS-6000 instrument. The IC instruments and methods were shown in Tables 35 and 36.

TABLE 35
IC instrument (anion)

| | |
|---|---|
| Model of the instrument | Thermo ICS-6000 |
| Work station | Chromeleon Workstation |
| KOH eluent generator | EGC 500 KOH |
| Suppressor | Dionx ASRS 300 4 mm |
| Guard column | Dionex IonPac ™ MAG11-HC(4*50 mm) |
| Chromatography column | Dionex IonPac ™ MAG11-HC(4*250 mm) |

TABLE 36
Parameters of IC analysis method (anion)

| | |
|---|---|
| Data acquisition frequency | 5.0 Hz |
| Conductivity cell temperature | 35° C. |
| Concentration of eluent | 30 mM |
| Working model of suppressor | External Mode |
| Suppressor current | 75 mA |
| Column temperature | 30° C. |
| Flow rate | 1.0 mL/min |
| Elution gradient | Isocratic elution |
| Run time | 10 min |
| Injection volume | 25 μL |
| External circulating water flow rate | 1.5 mL/min |

What is claimed is:

1. A pharmaceutically acceptable salt of compound A, wherein compound A has the following structure: 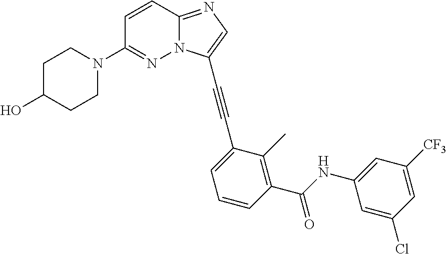 [SMILES: Cc1c(C#Cc2cnc3ccc(N4CCC(O)CC4)nn23)cccc1C(=O)Nc1cc(Cl)cc(C(F)(F)F)c1]
wherein the pharmaceutically acceptable salt is hydrochloride, sulfate, phosphate, p-toluenesulfonate, mesylate, besylate, oxalate, maleate, L-camphor sulfonate, gentianate, tartrate or fumarate.

2. The pharmaceutically acceptable salt of compound A of claim 1, wherein the pharmaceutically acceptable salt is phosphate.

3. The pharmaceutically acceptable salt of compound A of claim 2, wherein the phosphate is compound A phosphate crystalline form I, having at least one characteristic XRPD peak in terms of 2θ selected from 4.3°±0.2°, 17.4°±0.2°, 21.6°±0.2° and 21.9°±0.2°.

4. The pharmaceutically acceptable salt of compound A of claim 1, wherein the pharmaceutically acceptable salt is fumarate.

5. The pharmaceutically acceptable salt of compound A of claim 4, wherein the fumarate is compound A fumarate crystalline form I, having at least one characteristic XRPD peak in terms of 2θ selected from 9.5°±0.2°, 10.5°±0.2°, 14.3°±0.2° and 21.9°±0.2°.

6. A preparation method of compound A phosphate crystalline form I of claim 3, comprising the following steps:
contacting compound A with phosphoric acid in a solvent to form compound A phosphate;
precipitating compound A phosphate as compound A phosphate form I.

7. A preparation method of compound A fumarate crystalline form I of claim 5, comprising the following steps:
contacting compound A with fumaric acid in a solvent to form compound A fumarate;
precipitating compound A fumarate as compound A fumarate form I.

8. A pharmaceutical composition comprising a pharmaceutically acceptable salt of compound A of any one of claims 1 to 5.
